(19) United States
(12) Patent Application Publication
(10) Pub. No.: US 2017/0080125 A1
(43) Pub. Date: Mar. 23, 2017

(54) Methods of Promoting Bone Growth and Healing

(71) Applicant: The Penn State Research Foundation, University Park, PA (US)

(72) Inventor: Jian Yang, State College, PA (US)

(73) Assignee: The Penn State Research Foundation, University Park, PA (US)

(21) Appl. No.: 15/126,228

(22) PCT Filed: Mar. 17, 2015
(86) PCT No.: PCT/US15/20926

§ 371 Date: Sep. 14, 2016

Related U.S. Application Data
(60) Provisional application No. 61/954,156, filed on Mar. 17, 2014.

(51) Int. Cl. A61L 27/46; C08G 63/12; C08G 63/91; A61L 27/56

(52) U.S. Cl. CPC A61L 27/46; A61L 27/56; C08G 63/12; C08G 63/914; A61L 2430/02; A61L 2430/38

(57) ABSTRACT

In one aspect, methods of promoting bone growth are described herein. In some embodiments, a method described herein comprises disposing a graft or scaffold in a bone growth site. The graft or scaffold comprises (a) a polymer network formed from the reaction product of (i) citric acid, a citrate or an ester of citric acid with (ii) a polyol. The graft or scaffold further comprises (b) a particulate inorganic material dispersed in the polymer network.

### CROSS REFERENCE TO RELATED APPLICATIONS

This application claims priority pursuant to 35 U.S.C. §119 to U.S. Provisional Patent Application Ser. No. 61/954,156, filed on Mar. 17, 2014, which is hereby incorporated by reference in its entirety.

### STATEMENT REGARDING FEDERALLY SPONSORED RESEARCH

This invention was made with government support under Grant No. EB012575, awarded by the National Institutes of Health and under Grant No. DMR1313553, awarded by the National Science Foundation. The Government has certain rights in the invention.

### FIELD

This invention relates to methods and compositions for the promotion of bone growth, repair, and/or healing and, in particular, to methods of promoting bone growth, repair, and/or healing using graft or scaffold materials.

### BACKGROUND

Over 2.2 million bone transplantation procedures are performed worldwide in a variety of fields including orthopedics, neurosurgery, and dentistry. The bone autograft, the “gold standard” for bone grafting procedures, displays its limitations through its relatively high complication rate and prolonged recovery time. Its use has been associated with an 8-20% donor site complication rate that includes hematoma, soft tissue breakdown, pain, and a lengthened recovery time. Moreover, the use of bone autografts in osteoporotic populations is usually contra-indicated given a significant reduction in the quality and quantity of available bone. The ultimate tragedy, bone nonunion, can also result from the clinical implantation of ineffective materials, resulting in inflammation and scar formation.

Further, approximately 50% of bone transplantation procedures are spinal fusions, which have become a routine procedure in the field of spinal surgery for the treatment of cervical vertebra instability, lumbar degradation, invertebral disc injury, and spinal deformity diseases. Typically, spinal fusion surgery is effective in achieving vertebral stability and nerve decompression. However, the rates of fusion failure and pseudoarthrosis development have been reported as high as 5 to 35%. Conventionally, autologous bone remains the most widely used filling material for intervertebral fusion due to its non-immunogenic properties and high intervertebral fusion rates as compared to other materials. However, autologous bone use is limited due to an array of disadvantages including surgical trauma, increased risk of postoperative complications, and limited quantity of suitable autologous bones. Although the application of allograft and xenograft bones may solve the problem of limited supply while avoiding the additional surgical trauma associated with harvesting autologous bone, such materials introduce concerns of autoimmune response and an increased risk of bone disease spreading. Therefore, in light of the above-identified shortcomings, in combination with additional issues associated with prior methods and compositions, improved materials for promoting bone growth and/or repair are needed.

### SUMMARY

In one aspect, methods and compositions for the promotion of bone growth, healing, and/or repair are described herein which, in some embodiments, may provide one or more advantages compared to some other methods and compositions. For example, in some instances, methods and compositions described herein can provide reduced occurrence of donor site complications such as hematoma, soft tissue breakdown, pain, and/or lengthened recovery time. Further, in some cases, methods described herein can be used in osteoporotic populations where prior methods may be contra-indicated. Moreover, in certain instances, methods described herein can provide reduced occurrence of bone nonunion, thereby reducing occurrence of inflammation and/or scar formation at a donor site.

In some embodiments, a method of promoting bone growth described herein comprises disposing a graft or scaffold in a bone growth site, the graft or scaffold comprising (a) a polymer network formed from the reaction product of (i) citric acid, a citrate, or an ester of citric acid with (ii) a polyol. The graft or scaffold further comprises (b) a particulate inorganic material dispersed in the polymer network. In some embodiments, the particulate inorganic material comprises one or more of hydroxyapatite, tricalcium phosphate, biphasic calcium phosphate, bioglass, ceramic, magnesium powder, magnesium alloy, and decellularized bone tissue particles. In certain cases, the graft or scaffold further comprises a porous shell component and/or a porous core component. For example, in some embodiments, the graft or scaffold comprises a porous shell component surrounding a porous core component. In some such instances, the core component and the shell component are concentric cylinders.

In certain cases, the polymer network is formed from the reaction product of (i) citric acid, a citrate, or an ester of citric acid with (ii) a polyol and (iii) at least a third material or component. For example, in some instances, the polymer network is formed from the reaction product of (i) citric acid, a citrate, or an ester of citric acid with (ii) a polyol and (iii) an amine, an amide, or an isocyanate. In addition, in some embodiments, the polymer network is formed from the reaction product of (i) citric acid, a citrate, or an ester of citric acid with (ii) a polyol and (iii) a polycarboxylic acid or a functional equivalent of a polycarboxylic acid. Further, in some cases, the polymer network is formed from the reaction product of (i) citric acid, a citrate, or an ester of citric acid with (ii) a polyol and (iii) an amino acid. Moreover, in some instances, the polymer network is formed from the reaction product of (i) citric acid, a citrate, or an ester of a citric acid with (ii) a polyol and (iii) a catechol-containing species. In some embodiments, the polymer network is formed from (i) citric acid, a citrate, or an ester of citric acid with (ii) a polyol and (iii) at least one monomer comprising an alkyne moiety and/or an azide moiety. Further, in certain instances, the polymer network is formed from one or more monomers of Formula (A) hereinbelow, one or more monomers of Formula (B1) or (B2) hereinbelow, at least one monomer comprising an alkyne moiety and/or an azide moiety, and at least one monomer comprising an amine moiety and one or more hydroxyl moieties, such as a primary, secondary, or tertiary amine-containing diol.

Further, in some embodiments, a method described herein comprises additional steps or processes. For example, in some cases, methods described herein further comprise reestablishing a blood supply to the bone growth site and/or a biological region adjacent to the bone growth site. In certain instances, a method can comprise increasing one or more of osteoconduction, osteoinduction, and angionesis within the bone growth site and/or a biological area adjacent to the bone growth site. Moreover, in some embodiments, the method further comprises stimulating regeneration of bone and/or soft tissue proximate to the bone growth site. Additionally, methods described herein can comprise or include maintaining the graft or scaffold in the bone growth site for up to 6 months.

These and other embodiments are described in more detail in the detailed description which follows.

### BRIEF DESCRIPTION OF THE FIGURES

FIGS. 1A-1C illustrate radiographic images of a bone defect treated by a control method.

FIGS. 1D-1F illustrate radiographic images of a bone defect treated by a prior method of promoting bone growth.

FIGS. 1G-1I illustrate radiographic images of a bone defect treated by a method of promoting bone growth consistent with one embodiment described herein.

FIGS. 1J-1L illustrate radiographic images of a bone defect treated by a method of promoting bone growth consistent with one embodiment described herein.

FIGS. 2A and 2B illustrate charts of bone mineral density (BMD) and trabecular thickness (Tb.Th) of bone samples taken from test subjects treated by control methods of promoting bone growth, prior methods of promoting bone growth, and methods of promoting bone growth consistent with embodiments described herein.

FIG. 3 illustrates a chart of blood vessel development in test subjects treated by control methods of promoting bone growth, prior methods of promoting bone growth, and methods of promoting bone growth consistent with embodiments described herein.

FIG. 4A illustrates a reaction scheme for making a composition consistent with one embodiment described herein.

FIG. 4B illustrates a schematic representation of a method of making a graft or scaffold usable in methods consistent with embodiments described herein.

FIG. 5 illustrates a chart of bone fusion rate in test subjects treated by prior methods of promoting bone growth as well as methods of promoting bone growth consistent with embodiments described herein.

FIGS. 6A and 6B illustrate charts of data provided by biomechanical testing of test subjects treated by prior methods of promoting bone growth as well as methods of promoting bone growth consistent with embodiments described herein.

### DETAILED DESCRIPTION

Embodiments described herein can be understood more readily by reference to the following detailed description, examples, and figures. Elements, apparatus, and methods described herein, however, are not limited to the specific embodiments presented in the detailed description, examples, and figures. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

In one aspect, methods of promoting bone growth, healing, and/or repair are described herein. In some embodiments, a method of promoting bone growth comprises disposing a graft or scaffold in a bone growth site. A “graft” or “scaffold,” for reference purposes herein, can refer to any structure usable as a platform or implant for the replacement of missing bone or for promotion of growth of new bone. Moreover, as utilized herein, the terms “graft” or “scaffold” may be synonymous. For example, a graft or scaffold utilized in a method described herein can be used in the repair of a bone defect, the replacement of missing or removed bone, or for the promotion of new bone growth, as in the case of a bone fusion procedure. Further, it is to be understood that grafts or scaffolds consistent with methods described herein can have any structure or be formed in any shape, configuration, or orientation not inconsistent with the objectives of the present invention. For example, in some embodiments, a graft or scaffold can be shaped, configured, or oriented in such a manner as to correspond to a defect or bone growth site to be repaired. For example, a graft or scaffold utilized in the repair of a bone defect, such as a calvarial defect, may be formed, molded, or resized to a size and/or shape corresponding to the defect. In certain other cases, such as in a bone fusion procedure, a graft or scaffold utilized in methods described herein can have a shape, configuration, orientation, or dimensions adapted to traverse a gap between the bones to be fused and/or to reinforce a bone growth site. In this manner, particular shapes, sizes, orientations and/or configurations of grafts or scaffolds described herein are not intended to be limited to a particular set or subset of modalities on, within, or adjacent to a bone growth site. A “bone growth site,” as referenced herein, can be any area in which bone growth or repair may be desired. In certain non-limiting examples, a bone growth site can comprise or include a bone defect, a site in which bone has been removed or degraded, and/or a site of desired new bone growth, as in the case of a spinal or other bone fusion.

The graft or scaffold of a method described herein, in some cases, can comprise (a) a polymer network formed from the reaction product of (i) citric acid, a citrate, or an ester of citric acid with (ii) a polyol. The graft or scaffold can further comprise (b) a particulate inorganic material dispersed within the polymer network. The polymer network of a graft or scaffold described herein can comprise or be formed from any citrate-containing polymer not inconsistent with the objectives of the present invention. A “citrate-containing polymer,” for reference purposes herein, comprises a polymer or oligomer comprising a citrate moiety. In some cases, the citrate moiety is present in the backbone or main chain of the polymer. The citrate moiety may also be present in a pendant or side group or chain of the polymer. In some embodiments, the citrate moiety is a repeating unit of the polymer or is formed from a repeating unit of the polymer. Further, a “citrate moiety,” for reference purposes herein, comprises a moiety having the structure of Formula (I):

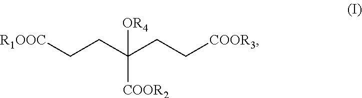

wherein R₁, R₂, and R₃ are independently —H, —CH₃, —CH₂CH₃, M⁺, or a point of attachment to the remainder of the polymer;
R₄ is —H or a point of attachment to the remainder of the polymer; and
M⁺ is a cation such as Na⁺ or K⁺, provided that at least one of R₁, R₂, R₃, and R₄ is a point of attachment to the remainder of the polymer.

For example, in some cases, a polymer of a composition described herein comprises the reaction product of (i) citric acid, a citrate, or an ester of citric acid, such as triethyl citrate or another methyl or ethyl ester of citric acid, with (ii) a polyol such as a diol. Non-limiting examples of polyols suitable for use in some embodiments described herein include C2-C20, C2-C12, or C2-C6 aliphatic alkane diols, including α,ω-n-alkane diols, or α,ω-alkene diols. For instance, in some cases, a polyol comprises 1,4-butanediol, 1,6-hexanediol, 1,8-octanediol, 1,10-decanediol, 1,12-dodecanediol, 1,16-hexadecanediol, or 1,20-icosanediol. Branched α,ω-alkane diols or α,ω-alkene diols can also be used. Additionally, a polyol can also be an aromatic diol. Further, in some embodiments, a polyol comprises a poly(ethylene glycol) (PEG) or polypropylene glycol) (PPG). Any PEG or PPG not inconsistent with the objectives of the present disclosure may be used. In some embodiments, for instance, a PEG or PPG has a weight average molecular weight between about 100 and about 5000 or between about 200 and about 1000.

A citrate-containing polymer of a graft or scaffold described herein, in some cases, can comprise the reaction product of (i) citric acid, a citrate, or an ester of citric acid with (ii) a polyol, and (iii) an amine, an amide, or an isocyanate. In such instances, the polyol can comprise any polyol described above, and the ester of citric acid can comprise any ester of citric acid described above. Further, an amine, in some embodiments, comprises one or more primary amines having two to ten carbon atoms. In other cases, an amine comprises one or more secondary or tertiary amines having two to fifteen carbon atoms. In some instances, an amine comprises a secondary or tertiary amine comprising one or more hydroxyl-containing groups bonded to the nitrogen. For example, in some cases, an amine comprises an amine-containing diol such as N-methyldiethanolamine (MDEA). An isocyanate, in some embodiments, comprises a monoisocyanate. In other instances, an isocyanate comprises a diisocyanate such as an alkane diisocyanate having four to twenty carbon atoms.

In addition, a citrate-containing polymer of a graft or scaffold described herein can also comprise the reaction product of (i) citric acid, a citrate, or an ester of citric acid with (ii) a polyol, and (iii) a polycarboxylic acid such as a dicarboxylic acid or a functional equivalent of a polycarboxylic acid, such as a cyclic anhydride or an acid chloride of a polycarboxylic acid. In such cases, the polyol can comprise any polyol described above, and the ester of citric acid can comprise any ester of citric acid described above. Moreover, the polycarboxylic acid or functional equivalent thereof can be saturated or unsaturated. For example, in some instances, the polycarboxylic acid or functional equivalent thereof comprises maleic acid, maleic anhydride, fumaric acid, or fumaryl chloride. A vinyl-containing polycarboxylic acid or functional equivalent thereof may also be used, such as allylmalonic acid, allylmalonic chloride, itaconic acid, or itaconic chloride. Further, in some cases, the polycarboxylic acid or functional equivalent thereof can be at least partially replaced with an olefin-containing monomer that may or may not be a polycarboxylic acid. In some embodiments, for instance, an olefin-containing monomer comprises an unsaturated polyol such as a vinyl-containing diol.

In still other embodiments, a citrate-containing polymer of a graft or scaffold described herein comprises the reaction product of (i) citric acid, a citrate, or an ester of citric acid with (ii) a polyol, and (iii) an amino acid such as an alpha-amino acid. Further, in some cases, a polymer described herein comprises the reaction product of (i) citric acid, a citrate, or an ester of citric acid with (ii) a polyol, (iii) an amino acid, and (iv) an isocyanate such as a diisocyanate. Additionally, in some instances, an acid anhydride and/or an acid chloride can be used in conjunction with the citric acid, citrate, or ester of citric acid. The polyol can be any polyol described above, the ester of citric acid can be any ester of citric acid described above, and the isocyanate can be any isocyanate described above. Further, the acid anhydride and/or acid chloride can include any acid anhydride and/or acid chloride described above, including, or instance, a polyacid anhydride or a polyacid chloride.

An alpha-amino acid of a polymer described herein, in some embodiments, comprises an L-amino acid, a D-amino acid, or a D,L-amino acid. In some cases, an alpha-amino acid comprises alanine, arginine, asparagine, aspartic acid, cysteine, glycine, glutamine, glutamic acid, histidine, isoleucine, leucine, lysine, methionine, proline, phenylalanine, serine, threonine, tyrosine, tryptophan, valine, or a combination thereof. Further, in some instances, an alpha-amino acid comprises an alkyl-substituted alpha-amino acid, such as a methyl-substituted amino acid derived from any of the 22 “standard” or proteinogenic amino acids, such as methyl serine. Additionally, in some cases, an amino acid forms a pendant group or side group of the polymer in a graft or scaffold utilized in a method described herein. Such an amino acid pendant group can be bonded to the backbone of the polymer in any manner not inconsistent with the objectives of the present disclosure. For example, in some cases, the amino acid is bonded to the backbone through an ester and/or amide bond between the amino acid and the citrate moiety. Moreover, in some instances, the amino acid forms a 6-membered ring with the citrate moiety. Not intending to be bound by theory, it is believed that the formation of a 6-membered ring described herein can provide fluorescence to the polymer. Thus, in some embodiments, the citrate-containing polymer a composition described herein can be a fluorescent polymer.

In addition, in some embodiments, a polymer described herein comprises the reaction product of (i) citric acid, a citrate, or an ester of a citric acid with (ii) a polyol, and (iii) a catechol-containing species. The citrate or ester of citric acid can be any citrate or ester of citric acid described above, such as a methyl or ethyl ester of citric acid. Similarly, the polyol can be any polyol described above, which may in some cases be referred to herein as a biodegradable photoluminescent polymer (BPLP).

The catechol-containing species can comprise any catechol-containing species not inconsistent with the objectives of the present disclosure. In some cases, a catechol-containing species used to form a citrate-containing polymer described herein comprises at least one moiety that can form an ester or amide bond with another chemical species used to form the polymer. For example, in some cases, a catechol-containing species comprises an amine moiety or a carboxylic acid moiety. Further, in some instances, a catechol-containing species comprises a hydroxyl moiety that is not part of the catechol moiety. In some embodiments, a catechol-containing species comprises dopamine. In other embodiments, a catechol-containing species comprises L-3,4-dihydroxyphenylalanine (L-DOPA) or D-3,4-dihydroxyphenylalanine (D-DOPA). In some cases, a catechol-containing species comprises 3,4-dihydroxyhydrocinnamic acid. Moreover, in some embodiments, a catechol-containing species is coupled to the backbone of the polymer through an amide bond. In other embodiments, a catechol-containing species is coupled to the backbone of the polymer through an ester bond.

A reaction product described hereinabove, in some cases, is a condensation polymerization reaction product of the identified species. Thus, in some embodiments, at least two of the identified species are comonomers for the formation of a copolymer. In some such embodiments, the reaction product forms an alternating copolymer or a statistical copolymer of the comonomers. Additionally, as described further herein, species described hereinabove may also form pendant groups or side chains of a copolymer.

In general, a citrate-containing polymer described herein can be any polymer or oligomer described in U.S. Pat. No. 7,923,486; U.S. Pat. No. 8,530,611; U.S. Pat. No. 8,574,311; U.S. Pat. No. 8,613,944; U.S. Patent Application Publication No. 2012/0322155; U.S. Patent Application Publication No. 2013/0217790; or U.S. Patent Application Publication No. 2014/066587; the entireties of which are hereby incorporated by reference. For example, in some cases, a citrate-containing polymer of a composition described herein comprises poly(ethylene glycol maleate citrate) (PEGMC), poly(octamethylene citrate) (POC), poly(octamethylene maleate anhydride citrate) (POMC), or a crosslinkable urethane doped elastomer (CUPE) or biodegradable photoluminescent polymer (BPLP).

In some embodiments, a citrate-containing polymer of a composition described herein is formed from one or more monomers of Formula (A) and one or more monomers of Formula (B1) or (B2):

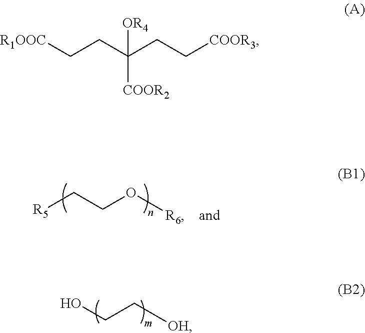

wherein R₁, R₂, and R₃ are independently —H, —CH₃, —CH₂CH₃, or M⁺;

### R₄ is —H;

R₅ is —H, —OH, —OCH₃, —OCH₂CH₃, —CH₃, or —CH₂CH₃;
R₆ is —H, —CH₃, or —CH₂CH₃;
M⁺ is a cation such as Na⁺ or K⁺; and
n and m are independently integers ranging from 1 to 20.
In some cases, for instance, R₁, R₂ and R₃ are —H, R₅ is —OH, and R₆ is —H.

Further, the monomers of Formula (A), (B1), and (B2) can be used in any ratio not inconsistent with the objectives of the present disclosure. In addition, altering the ratios of monomers can, in some embodiments, alter the biodegradability, the mechanical strength, and/or other properties of the polymer formed from the monomers. In some embodiments, the ratio of monomer (A) to monomer (B1) or monomer (B2) is between about 1:10 and about 10:1 or between about 1:5 and about 5:1. In some embodiments, the ratio of monomer (A) to monomer (B1) or monomer (B2) is between about 1:4 and about 4:1. In some embodiments, the ratio is about 1:1.

In some embodiments, a citrate-containing polymer of a graft or scaffold described herein has the structure of Formula (II):

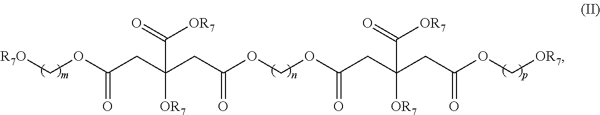

wherein each R₇ is independently —H or [symbol] ; [symbol]
represents an additional chain of repeating units having the structure of Formula (II); and
m, n, and p are independently integers ranging from 2 to 20.

In some embodiments, a polymer of a graft or scaffold utilized in a method described herein is formed from one or more monomers of Formula (A), one or more monomers of Formula (B1) or (B2), and one or more monomers of Formula (C):

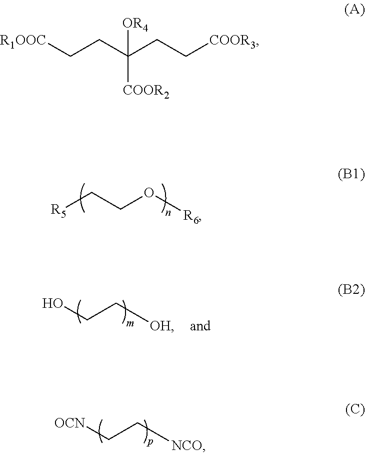

wherein R₁, R₂, and R₃ are independently —H, —CH₃, —CH₂CH₃, or M⁺;

### R₄ is —H;

R₅ is —H, —OH, —OCH₃, —OCH₂CH₃, —CH₃, or —CH₂CH₃;
R₆ is —H, —CH₃, or —CH₂CH₃;
M⁺ is a cation such as Na⁺ or K⁺;
n and m are independently integers ranging from 1 to 20; and
p is an integer ranging from 1 to 10.
For example, in some instances, R₁, R₂, and R₃ are —H, or —CH₂CH₃, R₅ is —OH, R₆ is —H, n is 2 to 6, m is 2 to 8, and p is 2 to 6.

Further, the monomers of Formula (A), (B1), (B2), and (C) can be used in any ratio not inconsistent with the objectives of the present disclosure. In addition, altering the ratios of monomers can, in some embodiments, alter the antimicrobial properties, the biodegradability, the mechanical strength, and/or other properties of the polymer formed from the monomers. In some embodiments, the ratio of monomer (A) to monomer (B1) or monomer (B2) is between about 1:10 and about 10:1 or between about 1:5 and about 5:1. In some embodiments, the ratio of monomer (A) to monomer (B1) or monomer (B2) is between about 1:4 and about 4:1. In some embodiments, the ratio is about 1:1. Further, in some embodiments, the ratio of monomer (A) to monomer (C) is between about 1:10 and about 10:1. In some embodiments, the ratio of monomer (A) to monomer (C) is about 1:1.

Further, in some embodiments described herein, a monomer of Formula (B1) or (B2) can be replaced by an alcohol that does not have the formula of Formula (B1) or (B2). For example, in some embodiments, an unsaturated alcohol or an unsaturated polyol can be used. Moreover, in some cases, a monomer of Formula (C) can be at least partially replaced by an amino acid described herein.

In some cases, a citrate-containing polymer of a graft or scaffold utilized in a method described herein comprises a polymer having the structure of Formula (III):

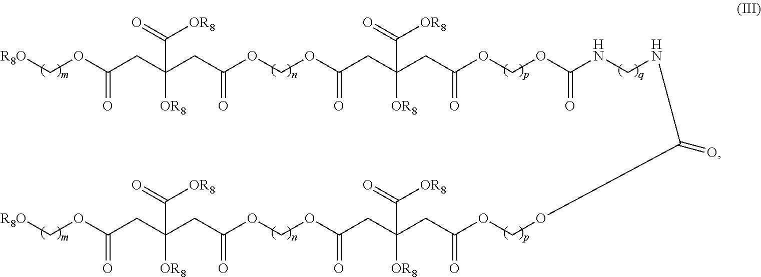

wherein
each R₈ is independently —H, —OC(O)NH [symbol] , or [symbol] ; [symbol]
represents an additional chain of repeating units having the structure of Formula (III); and
m, n, p, and q are independently integers ranging from 2 to 20.

In some embodiments, a citrate-containing polymer in a graft or scaffold utilized in a method described herein is formed from one or more monomers of Formula (A), one or more monomers of Formula (B1) or (B2), and one or more monomers of Formula (D) or (D′):

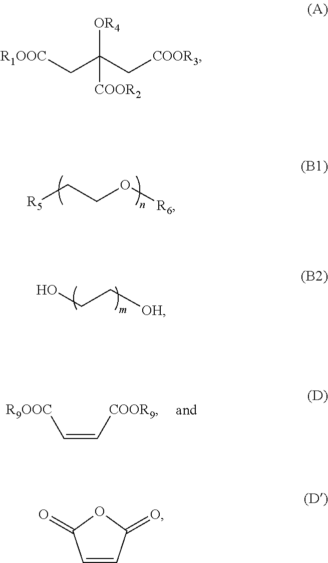

wherein R₁, R₂, and R₃ are each independently —H, —CH₃, —CH₂CH₃, or M⁺,

### R₄ is —H;

R₅ is —H, —OH, —OCH₃, —OCH₂CH₃, —CH₃, or —CH₂CH₃;
R₆ is —H, —CH₃, or —CH₂CH₃;
R₉ is —H, —CH₃, or —CH₂CH₃;
M⁺ is a cation such as Na⁺ or K⁺; and
n and m are each independently integers ranging from 1 to 20 or from 1 to 100.

Further, the monomers of Formula (A), (B1), (B2), (D) and (D′) can be used in any ratio not inconsistent with the objectives of the present disclosure. In addition, altering the ratios of monomers can, in some embodiments, alter the properties of the citrate-containing polymer formed from the monomers. In some embodiments, the ratio of monomer (A) to monomer (B1) or monomer (B2) is between about 1:10 and about 10:1 or between about 1:5 and about 5:1. In some embodiments, the ratio of monomer (A) to monomer (B1) or monomer (B2) is between about 1:4 and about 4:1. In some cases, the ratio is about 1:1. Further, in some embodiments, the ratio of monomer (A) to monomer (D) or monomer (D′) is between about 1:10 and about 10:1. In some embodiments, the ratio of monomer (A) to monomer (D) or monomer (D′) is about 1:1.

In some embodiments, a citrate-containing polymer in a graft or scaffold utilized in a method described herein comprises a polymer of Formula (IV):

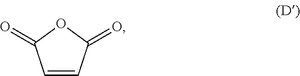

wherein R₁₀ is

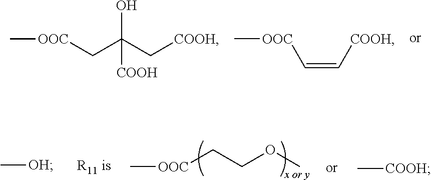

x and y are integers independently ranging from 1 to 100; and
z is an integer ranging from 1 to 20.

In some embodiments, a citrate-containing polymer of a graft or scaffold utilized in a method described herein is formed from one or more monomers of Formula (A) and one or more monomers of Formula (B1), (B2) or (B3), and one or more monomers of Formula (E):

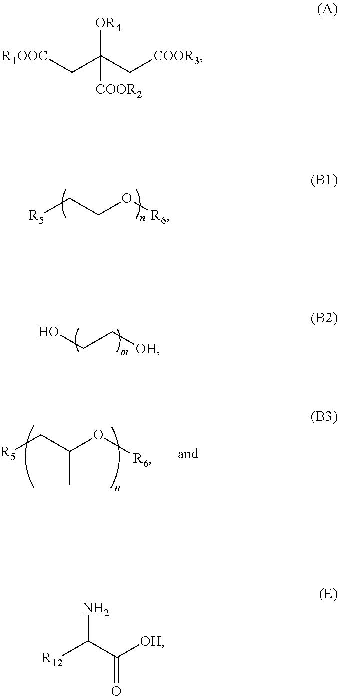

wherein R₁, R₂, and R₃ are independently —H, —CH₃, —CH₂CH₃, or M⁺;

### R₄ is —H;

R₅ is —H, —OH, —OCH₃, —OCH₂CH₃, —CH₃, or —CH₂CH₃;
R₆ is —H, —CH₃, or —CH₂CH₃;
R₁₂ is a side chain or “R group” of one of the 22 “standard” or proteinogenic amino acids provided above;
M⁺ is a cation such as Na⁺ or K⁺; and
n and m are independently integers ranging from 1 to 20.
In some cases, for example, R₁₂ is —CH₂SH (for E=cysteine) or —CH₂OH (for E=serine).
Further, in some embodiments, R₁, R₂, and R₃ are —H, R₅ is —OH, and R₆ is —H.

Moreover, the monomers of Formula (A), (B1), (B2), (B3) and (E) can be used in any ratio not inconsistent with the objectives of the present disclosure. In addition, altering the ratios of monomers can, in some embodiments, alter one or more properties of the citrate-containing polymer formed from the monomers. In some embodiments, the ratio of monomer (A) to monomer (B1), monomer (B2), or monomer (B3) is between about 1:10 and about 10:1 or between about 1:5 and about 5:1. In some embodiments, the ratio of monomer (A) to monomer (B1), monomer (B2), or monomer (B3) is between about 1:4 and about 4:1. In some cases, the ratio is about 1:1. Further, in some embodiments, the ratio of monomer (A) to monomer (E) is between about 1:10 and about 10:1.

In some embodiments, a polymer of a graft or scaffold described herein comprises a polymer of Formula (V):

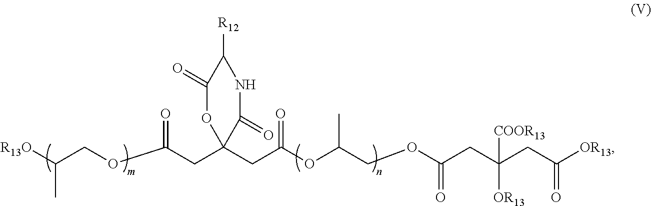

wherein R₁₂ is a side chain or “R group” of one of the 22 standard amino acids provided above;
each R₁₃ is independently —H or [symbol] ; [symbol]
represents an additional chain of repeating units having the structure of Formula (V); and

m and n are independently integers ranging from 2 to 20.

In some embodiments, a polymer of a composition described herein comprises a citrate-containing polymer formed from one or more monomers of Formula (A), one or more monomers of Formula (B1) or (B2), and one or more monomers of Formula (F):

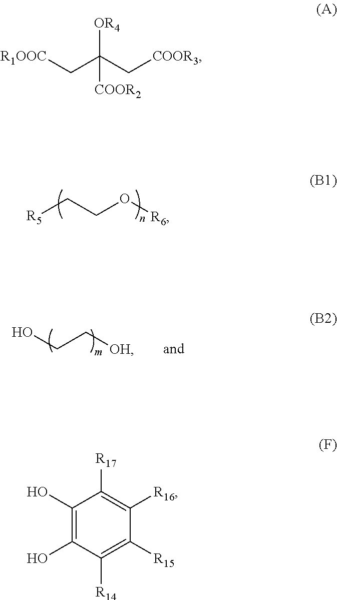

wherein R₁, R₂, and R₃ are independently —H, —CH₃, —CH₂CH₃, or M⁺;

### R₄ is —H;

R₅ is —H, —OH, —OCH₃, —OCH₂CH₃, —CH₃, or —CH₂CH₃;
R₆ is —H, —CH₃, or —CH₂CH₃;
R₁₄, R₁₅, R₁₆, and R₁₇ are independently —H, —CH₂(CH₂)ₓNH₂, —CH₂(CHR₁₈)NH₂, or —CH₂(CH₂)ₓCOOH;
R₁₈ is —COOH or —(CH₂)_{y}COOH;
M⁺ is a cation such as Na⁺ or K⁺;
n and m are independently integers ranging from 1 to 20;
x is an integer ranging from 0 to 20; and
y is an integer ranging from 1 to 20.

In some embodiments, R₂ is —H. In addition, in some cases, three of R₁₄, R₁₅, R₁₆, and R₁₇ are —H. Further, in some embodiments, R₁₄ and R₁₇ specifically are —H. In some cases, a monomer of Formula (F) comprises dopamine, L-DOPA, D-DOPA, or 3,4-dihydroxyhydrocinnamic acid. Moreover, in some embodiments, a monomer of Formula (F) is coupled to the backbone of the polymer through an amide bond. In other embodiments, a monomer of Formula (F) is coupled to the backbone of the polymer through an ester bond.

Further, in some embodiments, a monomer of Formula (B1) or (B2) can be replaced by an alcohol that does not have the formula of Formula (B1) or (B2). For example, in some embodiments, an unsaturated alcohol or an unsaturated polyol can be used.

Moreover, the monomers of Formula (A), (B1), (B2), and (F) can be used in any ratio not inconsistent with the objectives of the present disclosure. In addition, altering the ratios of monomers can, in some embodiments, alter one or more properties of the citrate-containing polymer formed from the monomers. In some embodiments, the ratio of monomer (A) to monomer (B1) or monomer (B2) is between about 1:10 and about 10:1 or between about 1:5 and about 5:1. In some embodiments, the ratio of monomer (A) to monomer (B1) or monomer (B2) is between about 1:4 and about 4:1. In some cases, the ratio is about 1:1. Further, in some embodiments, the ratio of monomer (A) to monomer (F) is between about 1:10 and about 10:1.

In some embodiments, a polymer of a graft or scaffold utilized in a method described herein comprises a polymer of Formula (VI):

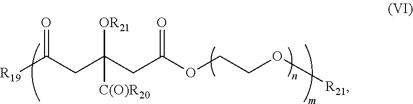

wherein R₁₉ and R₂₀ are independently

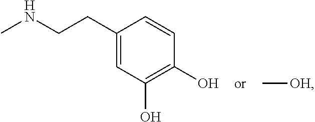

each R₂₁ is independently

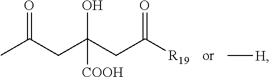

n is an integer ranging from 1 to 20, and
m is an integer ranging from 1 to 100, provided that at least one of R₁₉ and R₂₀ is

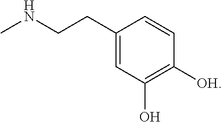

As described above, a citrate-containing polymer described herein can be a condensation polymerization reaction product of the identified monomers and/or other species. In some such embodiments, the reaction product forms an alternating copolymer or a statistical copolymer of the comonomers. Moreover, in some cases, the amount or ratio of a comonomer or other reactant comprising a citrate moiety can be selected to provide or tune a desired property or effect to the citrate-containing polymer. For example, in some embodiments, the amount or ratio of a comonomer or other reactant comprising a citrate moiety can be selected to provide a desired antimicrobial effect to the citrate-containing polymer. Other properties of a composition described herein can also be tuned by varying one or more of the mole percent or weight percent of a citrate moiety in a citrate-containing polymer. For example, tunable properties, in certain embodiments, can comprise or include one or more of: the antimicrobial properties of a citrate-containing polymer, the biodegradability of a citrate-containing polymer, and the water swellability of a citrate-containing polymer. In some cases, a citrate-containing polymer described herein comprises at least about 30 mole percent, at least about 40 mole percent, or at least about 50 mole percent citrate moiety, based on the total number of moles of the comonomers of the polymer. In some embodiments, a citrate-containing polymer described herein comprises between about 30 mole percent and about 70 mole percent, between about 30 mole percent and about 60 mole percent, between about 30 mole percent and about 50 mole percent, between about 35 mole percent and about 60 mole percent, between about 35 mole percent and about 55 mole percent, between about 40 mole percent and about 70 mole percent, between about 40 mole percent and about 60 mole percent, or between about 40 mole percent and about 55 mole percent citrate moiety, based on the total number of moles of the comonomers of the polymer. Similarly, in some cases, a citrate-containing polymer described herein comprises at least about 5 weight percent, at least about 10 weight percent, or at least about 15 weight percent, at least about 25 weight percent, at least about 30 weight percent, or at least about 40 weight percent citrate moiety, based on the total weight of the polymer. In some embodiments, a citrate-containing polymer described herein comprises between about 5 weight percent and about 80 weight percent, between about 5 weight percent and about 70 weight percent, between about 10 weight percent and about 80 weight percent, between about 10 weight percent and about 60 weight percent, between about 20 weight percent and about 80 weight percent, between about 20 weight percent and about 60 weight percent, between about 30 weight percent and about 80 weight percent, or between about 40 weight percent and about 70 weight percent citrate moiety, based on the total weight of the polymer.

Additionally, in some cases, one or more properties of a polymer may be tuned based on the amount of the citrate moiety as well as on one or more other features of the chemical structure of the polymer. Moreover, one or more properties may be tunable independently of one or more other properties. For example, in some cases, the water uptake and/or degradation rate of a polymer described herein can be tuned for a desired application. Such tunability can provide advantages to a composition of a graft or scaffold utilized in a method described herein. For instance, some previous compositions or scaffolds require incorporation of antibiotics or inorganic materials like silver nanoparticles to exhibit antimicrobial properties. Thus, a high swelling ratio of such compositions could lead to a “burst” release rather than a sustained release of bacteria-killing agents, thereby limiting the anti-infection applications of such compositions in grafts or scaffolds. In contrast, some citrate-containing polymers and compositions described herein can have decoupled swelling and antimicrobial properties. Therefore, the structure and chemical composition of some citrate-containing polymers and compositions described herein can be selected to satisfy other requirements, such as mechanical requirements, without the need to sacrifice antimicrobial performance, including long term antimicrobial performance.

Additionally, a citrate-containing polymer described herein can have at least one ester bond in the backbone of the polymer. In some cases, a polymer has a plurality of ester bonds in the backbone of the polymer, such as at least three ester bonds, at least four ester bonds, or at least five ester bonds. In some embodiments, a polymer described herein has between two ester bonds and fifty ester bonds in the backbone of the polymer. Polymers having one or more ester bonds in the backbone of the polymer can be hydrolyzed in a biological or other aqueous environment to release free citric acid or citrate, in addition to other components. Not intending to be bound by theory, it is believed that the presence of citric acid in a biological environment can contribute to pH reduction, which may depress the internal pH of bacteria and alter the permeability of the bacterial membrane by disrupting substrate transport.

Further, citrate-containing polymers having a structure described herein, in some cases, can be biodegradable. A biodegradable polymer, in some embodiments, degrades in vivo to non-toxic components which can be cleared from the body by ordinary biological processes. In some embodiments, a biodegradable polymer completely or substantially completely degrades in vivo over the course of about 90 days or less, about 60 days or less, or about 30 days or less, where the extent of degradation is based on percent mass loss of the biodegradable polymer, and wherein complete degradation corresponds to 100% mass loss. Specifically, the mass loss is calculated by comparing the initial weight (W₀) of the polymer with the weight measured at a pre-determined time point (Wₑ) (such as 30 days), as shown in equation (1):

$$Mass loss ( \% ) = \frac{( {W}_{0} - {W}_{t} )}{{W}_{0}} \times 100. \qquad ( 1 )$$

Additionally, in some embodiments, a polymer network comprising a citrate-containing polymer described herein can further comprise a crosslinker. Any crosslinker not inconsistent with the objectives of the present disclosure may be used. In some cases, for example, a crosslinker comprises one or more olefins or olefinic moieties that can be used to crosslink citrate-containing polymers comprising ethyleneically unsaturated moieties. In some embodiments, a crosslinker comprises an acrylate or polyacrylate, including a diacrylate. In other cases, a crosslinker comprises one or more of 1,3-butanediol diacrylate, 1,6-hexanediol diacrylate, glycerol 1,3-diglycerolate diacrylate, di(ethylene glycol) diacrylate, poly(ethylene glycol) diacrylate, polypropylene glycol) diacrylate, and propylene glycol glycerolate diacrylate. In still other instances, a crosslinker comprises a nucleic acid, including DNA or RNA. In some embodiments, a crosslinker comprises a “click chemistry” reagent, such as an azide or an alkyne. In some embodiments, a crosslinker comprises an ionic cross linker. For instance, in some cases, a citrate-containing polymer is crosslinked with a multivalent metal ion, such as a transition metal ion. In some embodiments, a multivalent metal ion used as a crosslinker of the polymer comprises one or more of Fe, Ni, Cu, Zn, or Al, including in the +2 or +3 state.

In addition, a crosslinker described herein can be present in a graft or scaffold in any amount not inconsistent with the objectives of the present invention. For example, in some embodiments, a crosslinker is present in a composition for a graft or scaffold in an amount between about 5 weight percent and about 50 weight percent, between about 5 weight percent and about 40 weight percent, between about 5 weight percent and about 30 weight percent, between about 10 weight percent and about 40 weight percent, between about 10 weight percent and about 30 weight percent, or between about 20 weight percent and about 40 weight percent, based on the total weight of the composition.

Thus, in some embodiments, a graft or scaffold utilized in a method described herein comprises a citrate-containing polymer that is crosslinked to form a polymer network. A polymer network, in some embodiments, comprises a hydrogel. A hydrogel, in some cases, comprises an aqueous continuous phase and a polymeric disperse or discontinuous phase. Further, in some embodiments, a crosslinked polymer network described herein is not water soluble.

One non-limiting class of crosslinked polymer networks described herein includes polymer networks that are crosslinked via one or more click chemistry reactions. For example, in some embodiments, polymers described herein can comprise or include the reaction product of (i) citric acid, a citrate, or an ester of citric acid, such as triethyl citrate or another methyl or ethyl ester of citric acid with (ii) a polyol such as a diol and (iii) a monomer comprising an alkyne moiety and/or an azide moiety. Any polyol described herein above can be used. Further, in some instances, the polyol can be at least partially replaced by an alcohol having only one hydroxyl group or by an amine or an amide. Further, in some cases, the polyol can be at least partially replaced by a polymer or oligomer having one or more hydroxyl, amine, or amide groups. Such a polymer or oligomer, in some instances, can be a polyester, polyether, or polyamide. Thus, in some embodiments, a composition described herein comprises the reaction product of (i) citric acid, a citrate, or an ester of citric acid with (ii) an alcohol, amine, amide, polyester, polyether, or polyamide, and (iii) a monomer comprising an alkyne moiety and/or an azide moiety.

In some cases, a composition utilized in a graft or scaffold in a method described herein can comprise a polymer formed from one or more monomers of Formula (A); one or more monomers of Formula (B4), (B5), or (B6); and one or more monomers comprising one or more alkyne moieties and/or one or more azide moieties:

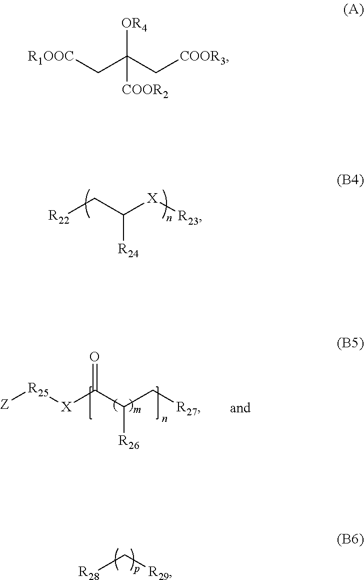

wherein
R₁, R₂, and R₃ are independently —H, —CH₃, —CH₂CH₃, or M⁺; R₄ is —H;
R₂₂ is —H, —OH, —OCH₃, —OCH₂CH₃, —CH₃, —CH₂CH₃, —NH₂, NHCH₃, —CH₂CH₂NHCH₃, —N(CH₃)₂, or —CH₂CH₂N(CH₂CH₃)₂;
R₂₃ is —H, —CH₃, or —CH₂CH₃, —(CH₃)₂, or —(CH₂CH₃)₂;
R₂₄ is —H or —CH₃;
R₂₅ is —(CH₂)ₐ—, —(CH₂CH₂O)_{b}— or —(CH₂OCH₂)_{b}—;
R₂₆ is —H, —CH₃, or a C2-C20 alkyl;
R₂₇ is —H, —C(O)CH₃, or —C(O)CH₂CH₃;
R₂₈ and R₂₉ are independently —OH or —NH₂;
M⁺ is a monovalent cation;
X and Y are independently —O— or —NH—;
Z is —H, —CH₃, —(CH₃)₂, —(CH₂CH₃)₂, or

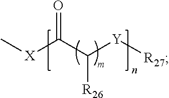

a is an integer from 0 to 20;
b is an integer from 0 to 2000;
n is an integer between 1 and 2000; and
m and p are independently integers ranging from 1 to 20; and
wherein the monomer of Formula (B4) has at least one terminus comprising —OH or —NH₂.

In some embodiments, one or more monomers of Formula (B4) is used, and X is —O—. Thus, in some cases, a monomer of Formula (B4) comprises

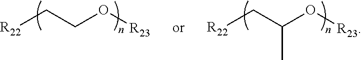

Further, in some instances, a monomer of Formula (B6) is used, and R₁₁ and R₁₂ are each —OH. In some embodiments, a monomer of Formula (B6) comprises

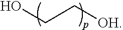

The monomers of Formula (A), (B4), (B5), and (B6) and the monomers comprising one or more alkyne and/or azide moieties can be used in any ratio not inconsistent with the objectives of the present disclosure. In addition, altering the ratios of monomers can, in some embodiments, alter the biodegradability, the mechanical strength, and/or other properties of the polymer formed from the monomers. In some embodiments, the ratio of monomer (A) to monomer (B4), (B5), or (B6) is between about 1:10 and about 10:1 or between about 1:5 and about 5:1. In some embodiments, the ratio of monomer (A) to monomer (B4), (B5), or (B6) is between about 1:4 and about 4:1. In some embodiments, the ratio is about 1:1. The ratio of an alkyne or azide-containing monomer to a monomer of Formula (A), (B4), (B5), or (B6) can be between about 1:20 and 1:2 or between about 1:10 and about 1:3. Further, a reaction product comprising one or more alkyne and/or azide moieties described herein, in some cases, is a condensation polymerization or polycondensation reaction product of the identified monomer or species.

Further, in some embodiments, a polymer of a composition described herein is formed from one or more additional monomers in addition to those recited above. For example, in some cases, a polymer of a composition described herein can comprise the reaction product of (i) citric acid, a citrate, or an ester of citric acid with (ii) a polyol, (iii) one or more alkynes and/or azides, and (iv) an amine, an amide, or an isocyanate. In such instances, the polyol can comprise any polyol described above, and the ester of citric acid can comprise any ester of citric acid described above. Further, the amine can comprise any amine described above, such as one or more primary amines having two to ten carbon atoms, one or more secondary or tertiary amines having two to fifteen carbon atoms, or one or more secondary or tertiary amines having one or more hydroxyl groups bonded to the nitrogen, as in the case of an amine-containing diol. The isocyanate can comprise any isocyanate described above. For example, in some embodiments, the polymer of a composition utilized in a graft or scaffold of a method described herein is formed from one or more monomers of Formula (A); one or more monomers of Formula (B4), (B5), or (B6); one or more monomers comprising one or more alkyne moieties and/or one or more azide moieties; and one or more monomers of Formula (G1), (G2), (G3), or (G4):

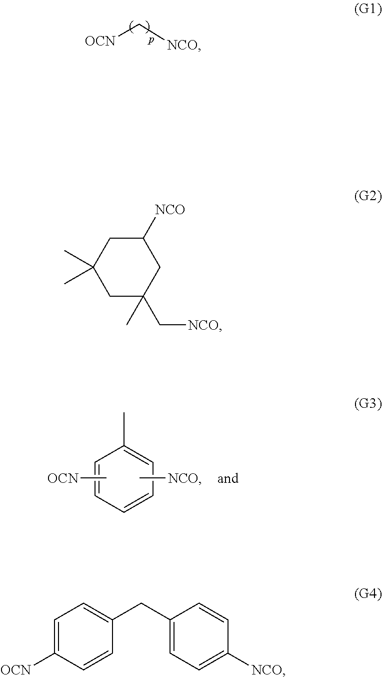

wherein
p is an integer ranging from 1 to 10.

Moreover, the monomers of Formula (A), (B4), (B5), (B6), (G1), (G2), (G3), and (G4) and the monomers comprising one or more alkyne and/or azide moieties can be used in any ratio not inconsistent with the objectives of the present disclosure. In addition, altering the ratios of monomers can, in some embodiments, alter the biodegradability, the mechanical strength, and/or other properties of the polymer formed from the monomers. In some embodiments, the ratio of monomer (A) to monomer (B4), (B5), or (B6) is between about 1:10 and about 10:1 or between about 1:5 and about 5:1. In some embodiments, the ratio of monomer (A) to monomer (B4), (B5), or (B6) is between about 1:4 and about 4:1. In some embodiments, the ratio is about 1:1. Further, in some embodiments, the ratio of monomer (A) to monomer (G) is between about 1:10 and about 10:1. In some embodiments, the ratio of monomer (A) to monomer (G1), (G2), (G3), or (G4) is about 1:1. The ratio of an alkyne or azide-containing monomer to a monomer of Formula (A), (B4), (B5), (B6), (G1), (G2), (G3), or (G4) can be between about 1:20 and 1:2 or between about 1:10 and about 1:3.

In addition, in some embodiments described herein, a monomer of Formula (B4), (B5), or (B6) can be replaced by an alcohol that does not have the formula of Formula (B4), (B5), or (B6). For example, in some embodiments, an unsaturated alcohol or an unsaturated polyol can be used. Moreover, in some cases, a monomer of Formula (G) can be at least partially replaced by an amino acid described herein.

Similarly, in other cases, a polymer comprises the reaction product of (i) citric acid, a citrate, or an ester of citric acid with (ii) a polyol, (iii) one or more alkynes and/or azides, and (iv) a polycarboxylic acid such as a dicarboxylic acid or a functional equivalent of a polycarboxylic acid, such as a cyclic anhydride or an acid chloride of a polycarboxylic acid. In such cases, the polyol can comprise any polyol described above, and the ester of citric acid can comprise any ester of citric acid described above. Moreover, the polycarboxylic acid can comprise any polycarboxylic acid or functional equivalent described above.

Further, the monomers of Formula (A), (B4), (B5), (B6), the polycarboxylic acid or functional equivalent, and the monomers comprising one or more alkyne and/or azide moieties can be used in any ratio not inconsistent with the objectives of the present disclosure. In addition, altering the ratios of monomers can, in some embodiments, alter the mechanical properties and/or other properties of the polymer formed from the monomers. In some embodiments, the ratio of monomer (A) to monomer (B4), (B5), or (B6) is between about 1:10 and about 10:1 or between about 1:5 and about 5:1. In some embodiments, the ratio of monomer (A) to monomer (B4), (B5), or (B6) is between about 1:4 and about 4:1. In some cases, the ratio is about 1:1. Further, in some embodiments, the ratio of monomer (A) to the polycarboxylic acid or functional equivalent is between about 1:10 and about 10:1. In some embodiments, the ratio of monomer (A) to polycarboxylic acid or functional equivalent is about 1:1. The ratio of an alkyne or azide-containing monomer to a monomer of Formula (A), (B4), (B5), (B6), or polycarboxylic acid or functional equivalent can be between about 1:20 and 1:2 or between about 1:10 and about 1:3.

In still other embodiments, the polymer of a composition described herein comprises the reaction product of (i) citric acid, a citrate, or an ester of citric acid with (ii) a polyol, (iii) one or more alkynes and/or azides, and (iv) an amino acid such as an alpha-amino acid. Further, in some cases, a polymer described herein comprises the reaction product of (i) citric acid, a citrate, or an ester of citric acid with (ii) a polyol, (iii) one or more alkynes and/or azides, (iv) an amino acid, and (v) an isocyanate such as a diisocyanate. Additionally, in some instances, an acid anhydride and/or an acid chloride can be used in conjunction with the citric acid, citrate, or ester of citric acid. The polyol can be any polyol described above, the ester of citric acid can be any ester of citric acid described above, the amino acid can be any amino acid described above, and the isocyanate can be any isocyanate described above. Further, the acid anhydride and/or acid chloride can include any acid anhydride and/or acid chloride described above, including, or instance, a polyacid anhydride or a polyacid chloride.

In some cases, the polymer of a composition described herein is formed from one or more of monomers of Formula (A); one or more monomers of Formula (B4), (B5), or (B6); one or monomers comprising one or more alkyne moieties and/or one or more azide moieties; and one or more monomers of Formula (E).

Moreover, the monomers of Formula (A), (B4), (B5), (B6), and (E) and the monomers comprising one or more alkyne and/or azide moieties can be used in any ratio not inconsistent with the objectives of the present disclosure. In addition, altering the ratios of monomers can, in some embodiments, alter the mechanical and/or other properties of the polymer formed from the monomers. In some embodiments, the ratio of monomer (A) to monomer (B4), monomer (B5), or monomer (B6) is between about 1:10 and about 10:1 or between about 1:5 and about 5:1. In some embodiments, the ratio of monomer (A) to monomer (B4), monomer (B5), or monomer (B6) is between about 1:4 and about 4:1. In some cases, the ratio is about 1:1. Further, in some embodiments, the ratio of monomer (A) to monomer (E) is between about 1:10 and about 10:1. The ratio of an alkyne or azide-containing monomer to a monomer of Formula (A), (B4), (B6), (B6), or (E) can be between about 1:20 and 1:2 or between about 1:10 and about 1:3.

In other instances, a polymer of a composition described herein comprises the reaction product of (i) citric acid, a citrate, or an ester of citric acid with (ii) a polyol, (iii) one or more alkynes and/or azides, and (iv) a catechol-containing species. The citrate or ester of citric acid can be any citrate or ester of citric acid described above, such as a methyl or ethyl ester of citric acid. Similarly, the polyol can be any polyol described above. Additionally, the catechol-containing species can comprise any catechol-containing species described herein above.

In some cases, a polymer of a composition described herein is formed from one or more monomers of Formula (A); one or more monomers of Formula (B1), (B2) or (B3); one or more monomers comprising one or more alkyne moieties and/or one or more azide moieties; and one or more monomers of Formula (F). Moreover, the monomers of Formula (A), (B4), (B5), (B6), and (F) and the monomers comprising one or more alkyne and/or azide moieties can be used in any ratio not inconsistent with the objectives of the present disclosure. In addition, altering the ratios of monomers can, in some embodiments, alter the mechanical properties and/or other properties of the polymer formed from the monomers. In some embodiments, the ratio of monomer (A) to monomer (B4), (B6), or (B6) is between about 1:10 and about 10:1 or between about 1:5 and about 5:1. In some embodiments, the ratio of monomer (A) to monomer (B4), (B5), or (B6) is between about 1:4 and about 4:1. In some cases, the ratio is about 1:1. Further, in some embodiments, the ratio of monomer (A) to monomer (F) is between about 1:10 and about 10:1. The ratio of an alkyne or azide-containing monomer to a monomer of Formula (A), (B4), (B5), (B6), or (F) can be between about 1:20 and 1:2 or between about 1:10 and about 1:3.

Monomers comprising one or more alkyne and/or azide moieties used to form a polymer described herein can comprise any alkyne- and/or azide-containing chemical species not inconsistent with the objectives of the present disclosure. For example, in some instances, one or more such monomers comprise a polyol such as a diol. Such a monomer, in some cases, can be incorporated into the polymer through the reaction of one or more hydroxyl moieties of the monomer with a carboxyl or carboxylic acid moiety of a monomer of Formula (A) or of another carboxyl-containing monomer described herein. Moreover, in some instances, such a monomer can be used instead of the monomer of Formula (B1) or (B2). In other instances, such a monomer is used in conjunction with one or more monomers of Formula (B1) or (B2). Further, such a monomer can be a diazido-diol (DAzD) or an alkyne diol (AlD).

In some cases, one or more monomers comprising one or more azide moieties comprises a monomer of Formula (H) or (H′):

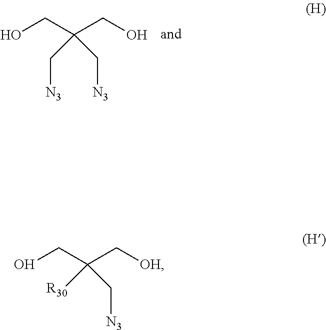

wherein
R₃₀ is —CH₃ or —CH₂CH₃.

Further, in some embodiments, one or more monomers comprising one or more alkyne moieties comprises a monomer of Formula (I1), (I2), (I3), (I4), (I5), or (I6):

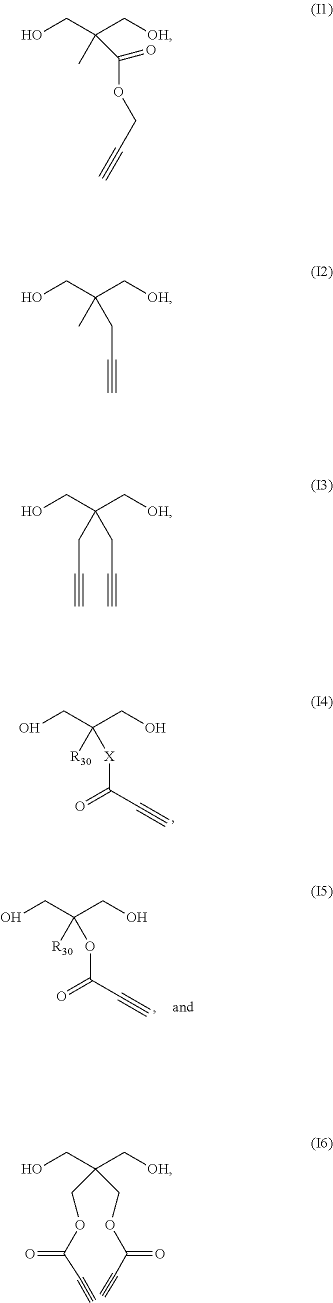

wherein
R₃₀ is —CH₃ or —CH₂CH₃; and

### X is —NH— or —O—.

Additionally, in some embodiments, a polymer described herein can be functionalized with a bioactive species. In some cases, the polymer is formed from an additional monomer comprising the bioactive species. Moreover, such an additional monomer can comprise one or more alkyne and/or azide moieties. For example, in some instances, a polymer described herein is formed from one or more monomers comprising a peptide, polypeptide, nucleic acid, or polysaccharide, wherein the peptide, polypeptide, nucleic acid, or polysaccharide is functionalized with one or more alkyne and/or azide moieties. In some cases, the bioactive species of a polymer described herein is a growth factor or signaling molecule. Further, a peptide can comprise a dipeptide, tripeptide, tetrapeptide, or a longer peptide. As described further hereinbelow, forming a polymer from such a monomer, in some embodiments, can provide additional biological functionality to a composition described herein.

In addition, in some embodiments, a composition comprises a plurality of polymers described herein. In some instances, the polymers are selected to be reactive with one another through a click chemistry reaction scheme. In some cases, for example, a composition described herein comprises a first polymer formed from one or more monomers of Formula (A); one or more monomers of Formula (B4), (B5), or (B6); and one or more monomers comprising one or more alkyne moieties; and further comprises a second polymer formed from one or more monomers of Formula (A); one or more monomers of Formula (B4), (B5), or (B6); and one or more monomers comprising one or more azide moieties. Thus, in some such embodiments, a composition described herein can comprise an azide-alkyne cycloaddition product, such as a 1,4 or 1,5-triazole ring. In this manner, a first polymer and a second polymer of a composition described herein can form a polymer network by forming one or more azide-alkyne cycloaddition products to serve as cross-links of the polymer network.

Such a polymer network can have a high cross-linking density. “Cross-linking density,” for reference purposes herein, can refer to the number of cross-links between polymer backbones or the molecular weight between cross-linking sites, calculated as described hereinbelow. Further, in some embodiments, the cross-links of a polymer network described herein comprise azide-alkyne cycloaddition product cross-links. Cross-links may also include ester bonds formed by the esterification or reaction of one or more pendant carboxyl or carboxylic acid groups with one or more pendant hydroxyl groups of adjacent polymer backbones. In some embodiments, a polymer network described herein has a cross-linking density of at least about 500, at least about 1000, at least about 5000, at least about 7000, at least about 10,000, at least about 20,000, or at least about 30,000 mol/m³. In some cases, the cross-linking density is between about 5000 and about 40,000 or between about 10,000 and about 40,000 mol/m³.

It is also possible to form a polymer network using a click chemistry reaction scheme that does not necessarily form azide-alkyne cycloaddition products. For instance, in some cases, one or more monomers comprising an alkyne and/or azide moiety described herein can be at least partially replaced by one or more monomers comprising a different moiety that can participate in a click chemistry reaction scheme. For example, in some embodiments, a polymer or polymer network is formed from the reaction of one or more monomers comprising a thiol moiety with one or more monomers comprising an alkene (or alkyne) moiety through a thiol-ene/yne click reaction. Such a thiol-ene/yne click reaction can comprise the addition of an S—H bond across a carbon-carbon double bond or triple bond by a free radical or ionic mechanism. More generally, in some cases, a polymer described herein can be formed from one or more monomers of Formula (A); one or more monomers of Formula (B4), (B5), or (B6); and one or more monomers comprising one or more first moieties operable to participate in a click chemistry reaction and/or one or more second moieties operable to participate in the same click chemistry reaction, where the first and second moieties differ. Any click chemistry reaction not inconsistent with the objectives of the present disclosure may be used. In some instances, the click chemistry reaction comprises a [3+2] cycloaddition such as a Huisgen alkyne-azide cycloaddition; a thiol-ene/yne reaction; a Diels-Alder reaction; an inverse electron demand Diels-Alder reaction; a [4+1] cycloaddition such as the cycloaddition reaction of an isocyanide with a tetrazine; or a nucleophilic substitution reaction involving a strained ring such as an epoxy or aziridine ring. Not intending to be bound by theory, it is believed that the use of a click chemistry reaction scheme to provide cross-linking in a polymer network can, in some cases, improve the mechanical strength of a polymer network without sacrificing pendant citric acid carboxyl moieties for other purposes, such as hydroxyapatite (HA) calcium chelation.

Further, it is to be understood that a polymer or polymer network described herein can be formed from monomers that are not necessarily monomers having the structure of Formula (A), (B4), (B5), or (B6). For example, in some cases, a polymer of a composition described herein is formed from one or more monomers comprising a lactone and one or more monomers comprising one or more moieties operable to participate in a click reaction, such as one or more alkyne moieties and/or one or more azide moieties. In some such cases, the one or more monomers comprising a lactone can comprise at least about 60 mol %, at least about 70 mol %, at least about 80 mol %, at least about 90 mol %, at least about 95 mol %, or at least about 99 mol % of the monomers used to form the polymer, based on the total amount of all monomers. Thus, in some instances, a polymer of a composition described herein comprises a polylactone that has been modified to include one or more clickable moieties such as one or more azide moieties and/or one or more alkyne moieties, including as pendant or side groups of the polymer. Any lactone not inconsistent with the objectives of the present disclosure may be used to form such a polymer. For example, in some cases, a lactone comprises L-lactide, D-lactide, D,L-lactide, glycolide, and/or ε-caprolactone. Thus, in some instances, a polymer described herein can be a poly(ε-caprolactone) (PCL), a poly(lactic-co-glycolic acid) (PLGA), or a combination thereof.

Similarly, in other embodiments, a polymer of a composition described herein is formed from one or more monomers comprising a polycarboxylic acid or a functional equivalent of a polycarboxylic acid that differs from a species described by Formula (A). Such a polycarboxylic acid can be a dicarboxylic acid, and a “functional equivalent” of a polycarboxylic acid can be a species that forms the same polymer product as a polycarboxylic acid does in a reaction scheme described herein, such as a cyclic anhydride or an acid chloride of a polycarboxylic acid described herein. Moreover, the polycarboxylic acid or functional equivalent thereof can be saturated or unsaturated. For example, in some instances, the polycarboxylic acid or functional equivalent thereof comprises maleic acid, maleic anhydride, fumaric acid, or fumaryl chloride. A vinyl-containing polycarboxylic acid or functional equivalent thereof may also be used, such as allylmalonic acid, allylmalonic chloride, itaconic acid, or itaconic chloride.

In some cases, a polymer is formed from one or more such monomers comprising a polycarboxylic acid or polycarboxylic acid equivalent; one or more monomers comprising a polyol; and one or more monomers comprising one or more clickable moieties, such as one or more alkyne moieties and/or one or more azide moieties. For instance, in some cases, the polycarboxylic acid comprises a dicarboxylic acid such as sebacic acid. Similarly, the polyol can comprise a diol such as a diol provided above or a triol such as glycerol. Further, in some such cases, the one or more monomers comprising one or more clickable moieties such as one or more alkyne and/or azide moieties can comprise up to about 40 mol %, up to about 30 mol %, up to about 20 mol %, up to about 10 mol %, up to about 5 mol %, or up to about 1 mol % of the monomers used to form the polymer, based on the total amount of all monomers. Thus, in some instances, a polymer of a composition described herein comprises a polyester such as poly(glycerol sebacate) (PGS) that has been modified to include one or more azide moieties and/or one or more alkyne moieties, including as a pendant or side group of the polymer.

Polymers such as citrate-containing polymers described herein can be prepared in any manner not inconsistent with the objectives of the present disclosure. In some cases, for instance, a polymer described herein is prepared by one or more polycondensation reactions. Further, in some embodiments, a polycondensation reaction can be followed by cross linking of the polymer. As described further herein, such cross linking can be thermal cross linking or photoinitiated cross linking such as ultraviolet (UV) cross linking.

Various components of compositions which may form part or all of a graft or scaffold utilized in a method of promoting bone growth have been described herein. It is to be understood that a composition according to the present disclosure can comprise any combination of components and features not inconsistent with the objectives of the present disclosure. For example, in some cases, a composition forming part or all of a graft or scaffold utilized in a method described herein can comprise a combination, mixture, or blend of polymers described herein. Additionally, in some embodiments, such a combination, mixture, or blend can be selected to provide a composition, graft or scaffold having any antimicrobial property, biodegradability, mechanical property, and/or chemical functionality described herein.

Further, one or more polymers such as one or more citrate-containing polymers can be present in a composition forming part or all of a graft or scaffold utilized in a method described herein in any amount not inconsistent with the objectives of the present disclosure. In some cases, a composition, graft or scaffold consists or consists essentially of the one or more polymers such as the one or more citrate-containing polymers. In other instances, a composition, graft or scaffold comprises up to about 95 weight percent, up to about 90 weight percent, up to about 80 weight percent, up to about 70 weight percent, up to about 60 weight percent, up to about 50 percent, or up to about 40 weight percent polymer, based on the total weight of the composition, graft or scaffold. In some embodiments, the balance of a composition, graft or scaffold described herein can be water, an aqueous solution, and/or a particulate material, as described further hereinbelow.

As described herein, grafts or scaffolds can further comprise a particulate material dispersed in the polymer network of the graft or scaffold. Any particulate material not inconsistent with the objectives of the present disclosure may be used. In some cases, the particulate material comprises one or more of hydroxyapatite, tricalcium phosphate (including α- and β-tricalcium phosphate), biphasic calcium phosphate, bioglass, ceramic, magnesium powder, magnesium alloy, and decellularized bone tissue particles. Other particulate materials may also be used.

In addition, a particulate material described herein can have any particle size and/or particle shape not inconsistent with the objectives of the present disclosure. In some embodiments, for instance, a particulate material has an average particle size in at least one dimension of less than about 1000 μm, less than about 800 μm, less than about 500 μm, less than about 300 μm, less than about 100 μm, less than about 50 μm, less than about 30 μm, or less than about 10 μm. In some cases, a particulate material has an average particle size in at least one dimension of less than about 1 μm, less than about 500 nm, less than about 300 nm, less than about 100 nm, less than about 50 nm, or less than about 30 nm. In some instances, a particulate material has an average particle size recited herein in two dimensions or three dimensions. Moreover, a particulate material can be formed of substantially spherical particles, plate-like particles, needle-like particles, or a combination thereof. Particulate materials having other shapes may also be used.

A particulate material can be present in a composition utilized in a graft or scaffold in of method described herein in any amount not inconsistent with the objectives of the present disclosure. For example, in some cases, a composition utilized in a graft or scaffold of a method described herein comprises up to about 70 weight percent, up to about 60 weight percent, up to about 50 weight percent, up to about 40 weight percent, or up to about 30 weight percent particulate material, based on the total weight of the composition. In some instances, a composition comprises between about 1 and about 70 weight percent, between about 10 and about 70 weight percent, between about 15 and about 60 weight percent, between about 25 and about 65 weight percent, between about 25 and about 50 weight percent, between about 30 and about 70 weight percent, between about 30 and about 50 weight percent, between about 40 and about 70 weight percent, or between about 50 and about 70 weight percent particulate material, based on the total weight of the composition. For example, in some cases, a composition comprising a polymer network described herein comprises up to about 65 weight percent hydroxyapatite.

Moreover, in some embodiments, a composition utilized in a graft or scaffold of a method described herein can comprise a high amount of particulate material, such as an amount up to about 70 weight percent, even when the polymers used to form the polymer network have a low weight average molecular weight, such as a weight average molecular weight of less than about 2000, less than about 1000, or less than about 500. For example, in some instances, a composition described herein comprises a polymer network formed from a polymer described herein having a weight average molecular weight of less than about 2000, less than about 1000, or less than about 500, and further comprises hydroxyapatite particles dispersed in the polymer network in an amount up to about 70 weight percent. Additionally, in some cases, the polymer network is not cross-linked or substantially cross-linked, other than by any cross-linking that may be provided by the hydroxyapatite particles.

Further, a particulate material described herein can be dispersed in a polymer network in any manner not inconsistent with the objectives of the present disclosure. In some embodiments, for instance, the particulate material is mixed or ground into the polymer network. In addition, a particulate material described herein, in some cases, can be chelated or otherwise bound by one or more pendant functional groups of the polymer network. For instance, in some cases, a composition comprises hydroxyapatite particles dispersed in a polymer network described herein, wherein the hydroxyapatite is chelated by one or more pendant functional groups of the polymer network. In some embodiments, one or more carboxyl moieties or one or more citrate moieties of the polymer network chelate one or more calcium-containing portions of the hydroxyapatite.

In some embodiments, a composition described herein comprises a biphasic polymeric scaffold. A “biphasic” scaffold, for reference purposes herein, can have a two-component structure, such as a core-shell structure, wherein the two components have differing chemical and/or mechanical properties. In some cases, for instance, a core-shell polymeric scaffold described herein comprises a core component having a first porosity; and a shell component surrounding the core component and having a second porosity, the second porosity differing from the first porosity. Additionally, in some such embodiments, the core component exhibits a higher porosity than the shell component. For example, in some cases, the first porosity is between about 30% and about 99% and the second porosity is between about 0% and about 99%. In some embodiments, the first porosity is between about 65% and about 75% and the second porosity is between about 0% and about 50% or between about 5% and about 50%. Such a pore structure, in some instances, can mimic the bimodal distribution of cancellous and cortical bone, respectively. Other porosity differences between the first porosity and second porosity are also possible. Moreover, in some instances, the core component can exhibit a lower porosity than the shell component. The porosity of a polymeric component can be measured in any manner not inconsistent with the objectives of the present disclosure. In some cases, for instance, porosity is measured by determining the bulk volume of the porous sample and subtracting the volume of the polymer network material. Other methods may also be used.

Additionally, the core component and/or the shell component can exhibit any range of pore sizes not inconsistent with the objectives of the present disclosure. In some cases, for instance, the core component and/or the shell component exhibits an average pore size of about 800 nm to about 1000 μm. In some embodiments, the core component and/or the shell component exhibits an average pore size of about 1 μm to about 800 μm, about 5 μm to about 500 μm, about 10 μm to about 1000 μm, about 10 μm to about 100 μm, about 50 μm to about 500 μm, about 100 μm to about 1000 μm, about 100 μm to about 500 μm, or about 500 μm to about 1000 μm.

Moreover, it is to be understood that both the core component and the shell component of a core-shell scaffold described herein can be formed from a composition described hereinabove. Any composition described hereinabove may be used for the core and shell components of a scaffold. Thus, in some cases, the core component comprises a first polymer network formed from a polymer described hereinabove, and the shell component comprises a second polymer network formed from a polymer described hereinabove. For example, in some instances, the core component comprises a first polymer network formed from one or more monomers of Formula (A) hereinabove; one or more monomers of Formula (B1) or (B2) hereinabove; one or more monomers comprising an alkyne moiety; and one or more monomers comprising an azide moiety. The shell component of such a scaffold can comprise a second polymer network formed from one or more monomers of Formula (A); one or more monomers of Formula (B1) or (B2); one or more monomers comprising an alkyne moiety; and one or more monomers comprising an azide moiety. The polymers of the first and second polymer networks can be the same or different in chemical composition.

Similarly, in other embodiments, the first polymer network and/or the second polymer network of a scaffold described herein comprises the reaction product of an amine, an amide, or an isocyanate with the one or more monomers of Formula (A), one or more monomers of Formula (B1) or (B2), and one or more monomers comprising one or more alkyne moieties and/or azide moieties. In some cases, the first polymer network and/or the second polymer network comprises the reaction product of a polycarboxylic acid or a functional equivalent of a polycarboxylic acid with the one or more monomers of Formula (A), one or more monomers of Formula (B1) or (B2), one or more monomers comprising an alkyne moiety, and one or more monomers comprising an azide moiety. The first polymer network and/or the second polymer network of a scaffold can also comprise the reaction product of an amino acid with the one or more monomers of Formula (A), one or more monomers of Formula (B1) or (B2), one or more monomers comprising an alkyne moiety, and one or more monomers comprising an azide moiety.

In addition, in some embodiments, a polymer network of a biphasic scaffold described herein can comprise a composite polymer network, including a composite polymer network described hereinabove. For example, in some cases, a particulate inorganic material is dispersed within the first polymer network and/or the second polymer network. Any particulate inorganic material not inconsistent with the objectives of the present disclosure may be used. In some instances, for example, the particulate inorganic material comprises hydroxyapatite. Further, as described hereinabove, a particulate inorganic material can be present in a polymer network in various amounts. In some cases, for instance, a particulate inorganic material is present in the first polymer network and/or the second polymer network of a biphasic scaffold described herein in an amount up to about 70 weight percent, based on the total weight of the first polymer network and/or the second polymer network, respectively.

Further, a core-shell scaffold described herein can have various core-shell architectures. In some embodiments, for instance, the core component and the shell component are concentric cylinders. In some such cases, the diameter of the core component is about 1 percent to about 90 percent of the diameter of the shell component. Other ratios of diameters are also possible. In addition, a biphasic scaffold described herein can have other structures as well, in addition to concentric cylinder core-shell structures.

Moreover, biphasic scaffolds described herein, in some instances, can be used for the promotion of bone growth in vivo. For example, in some cases, a citrate-based polymer-hydroxyapatite composite of a scaffold can provide an osteoconductive surface for bone regeneration and tissue integration, while the biphasic scaffold design can mimic the hierarchical organization of cancellous and cortical bone. Specifically, such a scaffold design, in some instances, can provide both the necessary porosity in the internal (or core) phase for tissue ingrowth and also the reduced porosity in the external (or shell) phase needed to meet mechanical demands for the repair of large segmental bone defects and/or for promotion of bone growth, such as in the case of spinal fusion. Therefore, such compositions, in some embodiments, can simulate both the compositional and architectural properties of native bone tissue and also provide immediate structural support for large segmental defects and/or other bone growth sites following implantation.

For instance, as described further hereinbelow, biphasic scaffolds described herein can be used in vivo for the repair of bone defects, such as calvarial defects. Further, scaffolds described herein can, in some cases, be used in the promotion of fusion of bones, such as in the case of spinal fusion. Such scaffolds can also exhibit good biocompatibility and extensive osteointegration with host bone. Further, biphasic scaffolds described herein, in some instances, significantly enhance the efficiency of new bone formation with higher bone densities in the initial stages after implantation. Compared to some other materials, biphasic scaffolds described herein can also exhibit increased flexural strength, interfacial bone ingrowth, and periosteal remodeling at early time points after implantation, such as time points prior to 15 weeks. For instance, in some cases, a scaffold described herein exhibits a compressive peak stress between about 1 MPa and about 45 MPa, between about 10 MPa and about 45 MPa, between about 20 MPa and about 45 MPa, between about 25 MPa and about 45 MPa, or between about 30 MPa and about 40 MPa. In addition, it is to be understood that the compressive strength of each portion of a scaffold can be controlled at least in part by varying the wall thickness and/or porosity of the given portion. A scaffold described herein can also exhibit an initial modulus between about 50 MPa and about 1500 MPa, between about 100 MPa and about 1500 MPa, between about 100 MPa and about 1000 MPa, between about 300 MPa and about 1500 MPa, between about 500 MPa and about 1500 MPa, between about 500 MPa and about 1000 MPa, between about 750 MPa and about 1500 MPa, or between about 750 MPa and about 1250 MPa. Moreover, a scaffold described herein can also exhibit a peak compressive strain at break between about 2% and about 5%, between about 2% and about 4%, or between about 3% and about 5%.

A graft or scaffold disposed in or at a bone growth site consistent with methods described herein, in some embodiments, can be disposed within bone growth site that is seeded with or contains a biofactor or seed cell. For example, in some embodiments, a graft or scaffold can be seeded with a biofactor or cell such as mesenchymal stem cells (MSCs). In certain other embodiments, a graft or scaffold can be disposed at a bone growth site in addition to or in combination with an autologous bone graft. Biofactor or cells utilized in combination with a graft or scaffold described herein may be isolated or sourced from any host or by any means not inconsistent with the objectives of the present invention. For example, in some embodiments, the biofactor or cells can be harvested or isolated from the individual receiving the graft or scaffold. In certain other embodiments, the biofactor or cells can be harvested or isolated from a different individual, such as a compatible donor. In some other cases, the biofactor or cells can be grown or cultured from an individual, either the graft or scaffold recipient or another compatible individual. In certain other cases, the graft or scaffold is unseeded with a biofactor or cell upon disposition within, on, or near the bone growth site. Non-limiting examples of seed cells that may be used in some embodiments described herein include mesenchymal stem cells (MSCs), bone marrow stromal cells (BMSCs), induced pluripotent stem (iPS) cells, endothelial progenitor cells, and hematopoietic stem cells (HSCs). Other cells may also be used. Non-limiting examples of biofactors that may be used in some embodiments described herein include bone morphogenetic protein-2 (BMP-2), transforming growth factor β3 (TGFβ3), stromal cell-derived factor-1α (SDF-1α), erythropoietin (Epo), vascular endothelial growth factor (VEGF), Insulin-like Growth Factor-1 (IGF-1), platelet derived growth factor (PDGF), fibroblast growth factor (BGF), nerve growth factor (NGF), neurotrophin-3 (NT-3), and glial cell-derived neurotrophic factor (GDNF). Other therapeutic proteins and chemical species may also be used.

Methods of promoting bone growth, in some embodiments, can also comprise or include additional steps. Individual steps may be carried out in any order or in any manner not inconsistent with the objectives of the present disclosure. For example, in some embodiments, methods described herein further comprise reestablishing a blood supply to the bone growth site and/or a biological region adjacent to the bone growth site. In certain cases, reestablishing a blood supply can comprise or include sealing or suturing biological tissue adjacent to the bone growth site. Additionally, in some cases, where blood flow has been artificially restricted at or adjacent to the bone growth site, such as by clamping or suction, reestablishing a blood supply can comprise or include releasing or removing the artificial restriction. Further, in some cases, a method of promoting bone growth can comprise or include increasing one or more of osteoconduction, osteoinduction, osteogenesis, and angiogenesis within the bone growth site and/or a biological area adjacent to the bone growth site. Additionally, in some instances, methods further comprise stimulating regeneration of bone and/or soft tissue proximate the bone growth site.

Moreover, in some embodiments, methods of promoting bone growth described herein can comprise maintaining the graft or scaffold in the bone growth site for a period of time after disposing the graft or scaffold in the bone growth site. Any period of time not inconsistent with the objectives of the present disclosure can be used. For example, in some cases, the graft or scaffold can be maintained for at least 1 month, such as for at least 3 months, at least 6 months, at least 9 months, or at least 12 months. In certain embodiments, a graft or scaffold may degrade or biodegrade within the bone growth site. In such embodiments, maintenance of the graft or scaffold can comprise or include maintaining the graft or scaffold until a desired portion of the graft or scaffold has degraded or biodegraded. For example, methods can comprise maintaining the graft or scaffold in the bone growth site until at least 50%, at least 60%, at least 70%, at least 80%, at least 90%, at least 95%, or at least 99% of the graft or scaffold has degraded or biodegraded. In certain embodiments, methods can comprise maintaining the graft or scaffold in the bone growth site until all or substantially all of the graft or scaffold has degraded or biodegraded.

Some embodiments described herein are further illustrated in the following non-limiting examples.

### Example 1

### Polymer Networks

Polymer networks suitable for use in some embodiments of methods of promoting bone growth or repair described herein were prepared as follows. Specifically, poly(1,8-octanediol-co-citric acid) (POC) synthesis was carried out according to the following method.

Equimolar amounts of citric acid and 1,8-octanediol were added to a 250 mL three-neck round-bottom flask fitted with an inlet and outlet adapter. The mixture was melted under a flow of nitrogen gas by stirring at 160-165° C. in a silicon oil bath. The temperature was then lowered to 140° C. The mixture was stirred for an hour at 140° C. to create a “pre-polymer” solution. The pre-polymer solution was post-polymerized at 37° C., 60° C., 80° C., or 120° C. under vacuum (2 Pa) or at ambient pressure for times ranging from 1 day to 2 weeks to create POC with various degrees of cross-linking.

In addition to POC, several other citric acid based elastomers were synthesized as described above using other diols. The resulting copolymers were poly(1,6-hexanediol-co-citric acid) (PHC), poly(1,10-decanediol-co-citric acid) (PDC), and poly(1,12-dodecanediol-co-citric acid) (PDDC).

### Example 2

### Polymer Network

A polymer network suitable for use in some embodiments of methods of promoting bone growth or repair described herein was prepared as follows. Specifically, crosslinkable urethane doped elastomer (CUPE) synthesis was carried out according to the following method.

First, POC was synthesized according to Example 1 above with slight modifications. Briefly, citric acid and 1,8-octanediol, with a monomer ratio of 1:1.1, were bulk polymerized in a three-neck reaction flask, fitted with an inlet and outlet adapter at 160-165° C. Once the mixture had melted, the temperature was lowered to 140° C., and the reaction mixture was stirred for an hour to create the POC pre-polymer. The pre-polymer was purified by drop-wise precipitation in deionized water. Undissolved pre-polymer was collected and lyophilized to obtain the purified POC pre-polymer. The average molecular weight of pre-POC was characterized as 850 Da by matrix assisted laser desorption/ionization mass spectroscopy (MALDI-MS), which was performed using an Autoflex MALDI-TOF Mass Spectrometer (Bruker Daltonics, Manning Park, Mass.). Chain extension of the POC pre-polymer to obtain pre-CUPE was then performed.

Purified pre-POC was dissolved in 1,4-dioxane to form a 3 wt.-% solution (based on the total weight of the pre-POC and 1,4-dioxane). The polymer solution was then reacted with 1,6-hexamethyl diisocyanate (HDI) in a clean reaction flask under constant stirring, with stannous octoate as a catalyst (0.1 wt.-%). Different pre-CUPE polymers were synthesized with different feeding ratios of pre-POC:HDI (1:0.56, 1:0.9 and 1:1.2, molar ratio). The system was maintained at 55° C. throughout the reaction. Small amounts of the reaction mixture were removed at 6 hour intervals and subjected to Fourier transform infrared (FT-IR) analysis. The reaction was terminated when the isocyanate peak at 2267 cm⁻¹ disappeared.

The pre-CUPE solution was then cast in a TEFLON® (commercially available from DuPont) mold and allowed to dry in a chemical hood equipped with a laminar airflow until all the solvents had evaporated. The resulting pre-CUPE film was moved into an oven maintained at 80° C. for pre-determined time periods to obtain crosslinked CUPE.

### Example 3

### Scaffolds

Scaffolds suitable for use in some embodiments of methods of promoting bone growth or repair described herein were prepared as follows.

First, sodium chloride crystals were ground and separated by sieve into differing sizes ranging from 50 μm to 1000 μm. Hydroxyapatite (HA) and a pre-polymer, such as poly(1,8-octanediol) (POC) consistent with Example 1, crosslinkable urethane doped elastomer (CUPE) consistent with Example 2, or other citrate-based polymers were dissolved in 1,4-dioxane solvent at HA ratios (relative to the combined weight of HA and polymer) of 0 to 65 wt.-% under continuous stirring in a TEFLON® (commercially available from DuPont) dish. Next, salt was added to the above solutions with a salt weight ratio (based on the total weight of salt, polymer, and HA) from 0 to 90 wt.-%. The resulting slurry was stirred until nearly all of the solvent evaporated. The mixture was then transferred to molds.

Specifically, scaffolds were made using a cylindrical TEFLON® mold with a 4 mm inner diameter. After molding and ejection from the mold, all scaffolds were crosslinked at 80-100° C. for 3 days. After crosslinking, salt was leached out by immersing the scaffolds in distilled water. The salt leaching process was carried out for a time period up to 3 weeks, with water changes made at least every other day. Frequent water changes, applying vacuum and heating, or using a swelling solvent like ethanol could be used to reduce the time needed to remove all of the salt from the scaffolds. After salt leaching, scaffold samples were freeze-dried and sterilized before their use in animal studies.

### Example 4

### Methods of Promoting Bone Growth

Methods of promoting bone growth or healing according to some embodiments described herein were carried out as follows.

### Disposing Scaffolds in Bone Growth Sites

Seventy-two male Sprague-Dawley rates (200-220 g) were used in experiments. The body weights of the experimental animals were closely monitored to confirm feeding and expected growth rates. All rats were handled regularly for at least one week prior to surgery and individually housed in cages in climate controlled rooms at 22° C. with 50% humidity and 12 h light/dark cycles. Surgeries were performed under semi-sterile conditions with animals under anesthesia induced by the intraperitoneal injection of 100 mg/kg chloral hydrate and 10 mg/kg xylazine. The surgical site was shaved and prepared with a 70% ethanol solution. Sterile drapes, gloves, and instruments were used and the punch was disinfected with alcohol.

The rats were injected subcutaneously with 0.5 mL of 1% lidocaine (local anesthetic) at the sagittal midline of the skull. Following this injection, a sagittal incision was made over the scalp from the nasal bone to the middle sagittal crest and the periosteum was bluntly dissected. Using a punch, a 4 mm diameter pit defect was made with a trephine constantly cooled with sterile saline. The calvarial disk was then carefully removed to avoid tearing of the dura. After thorough rinsing with physiological saline to wash out any bone fragments, a composite scaffold was implanted within the defect. Animals were split equally into four treatment groups (n=18 per group): 1) a group in which a 4 mm defect was created but left empty (control group/CON group), 2) a group in which the defect was filled with autogenous bone (AB group), 3) a group in which a CUPE-HA scaffold according to Examples 2 and 3 was placed in the defect site (CUPE-HA group), and 4) a group in which a POC-HA scaffold according to Examples 1 and 3 was placed into the defect site (POC-HA group). The skin was sutured with 6-0 vicryl and animals were monitored using post-operative animal care protocols.

### Postoperative Care

All rats survived to the end of surgery and returned to normal activity within 24 hours. Following the surgical procedures, no signs of local infection, lingering algesia, or abnormal body weight fluctuations were observed. After 6 months of surgical implantation, the calvarial samples were harvested for gross and SEM observation.

Six rats died during the postoperative period, including one in the control group, two in the AB group, one in the CUPE-HA group, and two in the POC-HA group. The remaining 66 animals experienced an uncomplicated postoperative course until six months post-surgery. All rats were grossly observed daily. There were no obvious inflammatory responses in the animals.

### Radiographic Assessment of New Bone Formation

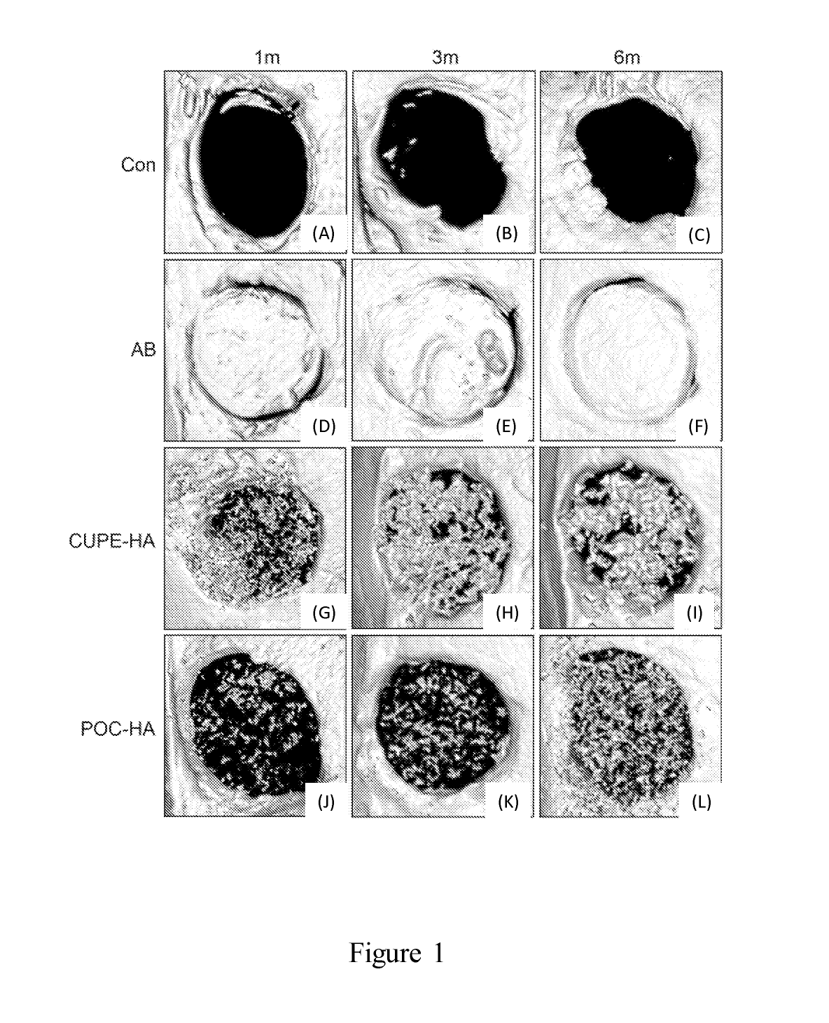

To observe new bone formation within the defects, 3D morphology images were reconstructed using micro-CT at 1, 3, and 6 months post-surgery. These images are illustrated in FIGS. 1A-1L. Radiographic evidence of new bone formation was highly variable between non-scaffold groups (the control and AB groups) and scaffold groups (CUPE-HA and POC-HA groups). In the non-scaffold groups, new bone formation was seen through the appearance of marginal growth. However, in the scaffold groups, new bone formation was spread all over the defects and radiopaque, especially in the CUPE-HA group illustrated in FIGS. 1G-1I.

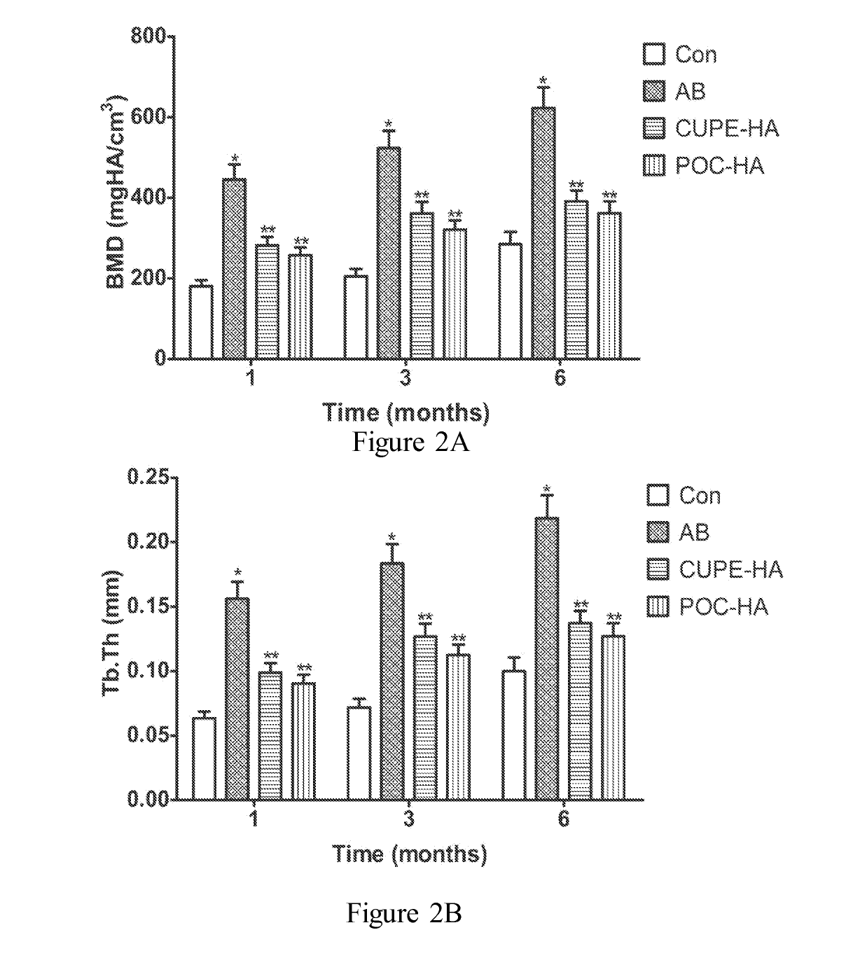

Micro-CT analysis was carried out next to provide further quantitative assessment of mineralized skeletal tissue formation at the edge of the defect site. The most apparent difference observed was the greater amount of newly formed bone in the AB group compared to lesser amount of bone formation in the other groups. Compared to the CON group, the two scaffold-treatment groups exhibited a greater repair effect, although less of an effect compared to the AB group. The bone mineral density (BMD) of the AB group in the areas undergoing repair at 1, 3, and 6 months after surgery was significantly higher than that of the other groups. Furthermore, the BMD of the CUPE-HA and POC-HA groups was higher than the CON group, as illustrated in FIG. 2. The trabecular thickness (Tb.Th) of each group showed a similar trend. No significant differences were observed between the CUPE-HA group and the POC-HA group.

### Histological Assessment

Histological findings for defect sites in the CUPE-HA and POC-HA groups were similar to findings in the AB group. Specifically, the edge of the defect site was composed of fibrous stroma and reactive bone, with the fibrous stroma appearing loose around the scaffolds and exhibiting a relatively high level of angiogenesis. In contrast, the defect sites in the CON group seldom exhibited reactive bone, with less fibrous stroma compared to the AB and both scaffold groups, as illustrated in FIGS. 2.

At 1, 3, and 6 months after implantation, immunohistochemical staining for vascular endothelial growth factor b (VEGF-b) was performed. In the CON group, the defect space (without implantation) was filled with small fibrous and inflammatory cells, and those cells were negative for VEGF-b. In contrast, the CUPE-HA and POC-HA groups showed more positive VEGF-b expression in the defect site, similar to the AB group.

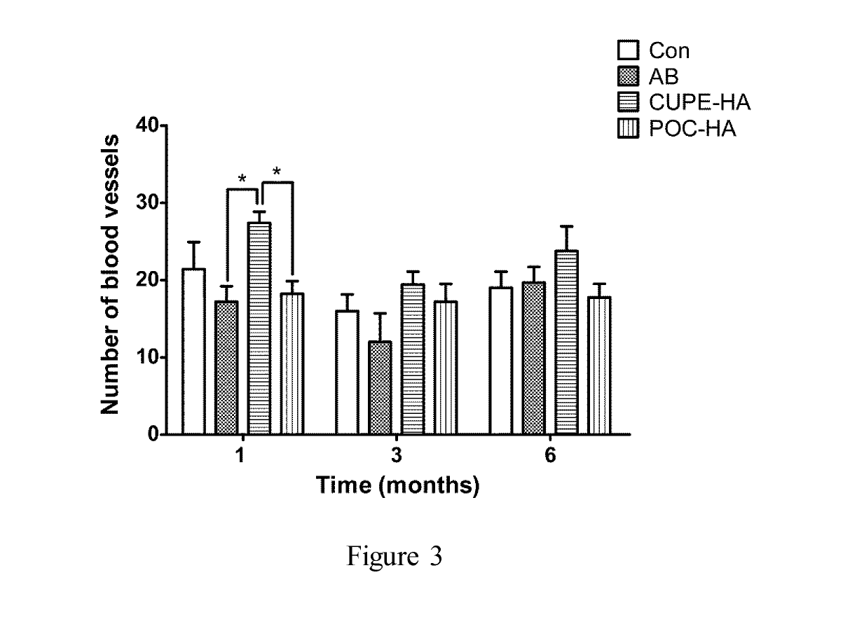

Boosted vessel numbers in scaffold-treated rats were confirmed by morphometric analysis of the vessels. The vascular numbers of CUPE-HA groups were significantly higher than the AB or POC-HA group at 1 month post-surgery, as illustrated in FIG. 3. In addition, there were no significant differences in vascular numbers between each group at 3 and 6 months post-surgery.

### Example 5

### Polymers

Polymer networks suitable for use in some embodiments of methods of promoting bone growth or repair described herein were prepared as follows. Specifically, polymers were formed using amine-containing diol monomers such as N-methyldiethanolamine (MDEA). The use of a monomer such as MDEA can increase the mechanical strength of a graft or scaffold described herein while maintaining certain other desirable characteristics such as degradability and/or biodegradability.

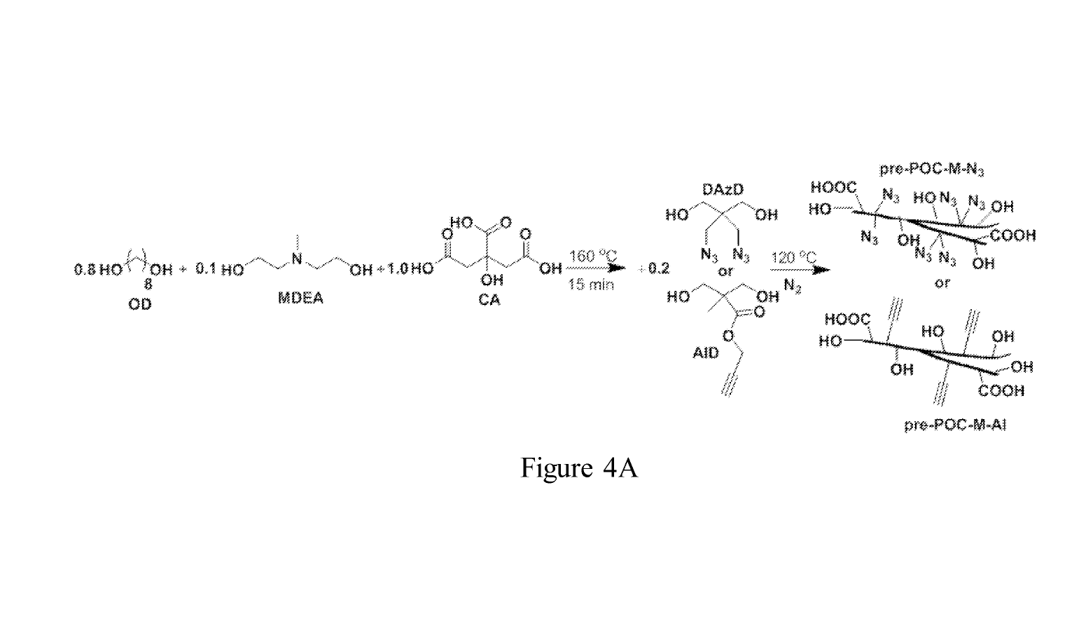

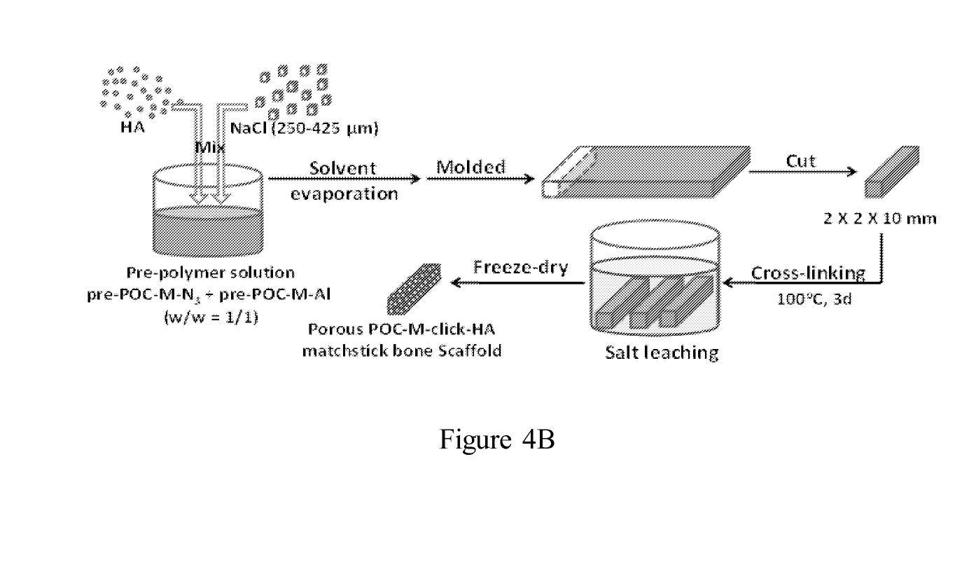

N-methyldiethanolamine (MDEA) modified poly(1,8-octanediol citrate)-click (POC-M-click) pre-polymers, containing MDEA, modified pre-POC-N₃, and pre-POC-Al (pre-POC-M-N₃ and pre-POC-M-A1) were synthesized as illustrated in FIG. 4A.

Briefly, for pre-POC-M-N₃, a mixture of citric acid (CA), 1,8-octanediol (OD), and MDEA at a molar ratio of CA:OD:MDEA of 1:0.8:0.1 was melted at 160° C. for 15 minutes. Afterwards, the reaction temperature was reduced to 120° C., followed by the addition of diazido-diol monomers (DAzD, 2,2-bis(azidomethyl)propane-1,3-diol) at a molar ratio of CA:DAzD of 1:0.2. The reaction was continued at 120° C. for approximately 2 hours. The crude product was then purified by precipitating the oligomer/1,4-dioxane solution in water, followed by freeze-drying to obtain pre-POC-M-N₃.

Pre-POC-M-Al was synthesized by reacting CA, OD, MDEA, and an alkyne diol monomer (AlD, 2,2-bis(hydroxyl-methyl)priopionate) in place of DAzD at a molar ratio of CA:OD:MDEA:AlD of 1:0.8:0.1:0.2 using similar protocols to those used above for pre-POC-M-N₃.

### Example 6

### Scaffolds

Scaffolds suitable for use in some embodiments of methods of promoting bone growth or repair described herein were prepared as follows.

Porous POC-M-click-HA composite matchstick-shaped scaffolds, with a size of 2×2×10 mm, an HA content of 65 wt.-% relative to the combined weight of HA and polymer, a porosity of 65%, and pore size of 250-425 μm were fabricated according to the following method. Scaffolds were prepared according to the method illustrated in FIG. 4B utilizing the compositions prepared according to Example 5 above.

Sodium chloride particles with a size of 250-425 μm were used as a porogen. Sodium chloride particles were first bonded together using polyvinylpyrrolidone (PVP, with a M_{w} of 10 KDa) (commercially available from Sigma-Aldrich). PVP (10 vol.-% relative to the combined amount of salt and PVP) was dissolved in ethanol and the solution was mixed with salt and stirred continuously until the ethanol evaporated. A POC-M-click pre-polymer solution was prepared from an equal weight mixture of pre-POC-M-N₃ and pre-POC-M-Al dissolved in 30 wt.-% 1,4-dioxane solvent. After salt bonding, a 65 wt.-% solution of HA and POC-M-click pre-polymer solution (65 wt.-% HA relative to the combined weight of HA and pre-polymer) was mixed and stirred continuously until nearly all solvent was evaporated. The mixture was kneaded by hand until the composite was sufficiently dry, maintaining desired workability.

Matchstick bone scaffolds were then made in cuboid TEFLON® (commercially available from DuPont) molds with a size of 104×2×10 mm. After drying, the large scaffold was cut to the desired size of 2×2×10 mm and crosslinked at 100° C. for 3 days to perform a synchronous dual crosslinking process, namely thermal click reaction and esterification. After cross-linking, salt and PVP were leached out by immersing the scaffolds in deionized water. After salt/PVP leaching, scaffold samples were freeze-dried and sterilized. Porous poly(L-lactic acid)-HA (PLLA-HA, PLLA with a M_{w}˜60 KDA, purchased from Polyscitech) matchstick scaffolds of the same size (2×2×10 mm), porosity (65%), HA content (65 wt.-%) and pore size (250-425 μm) as the POC-M-click-HA scaffolds were also prepared to serve as controls in animal studies.

### Example 7

### Methods of Promoting Bone Growth

Methods of promoting bone growth or healing according to some embodiments described herein were carried out as follows.

### Disposing Scaffolds in Bone Growth Sites

A set of 54 rabbits (average weight, 2-2.5 kg, male or female) were randomly divided into three groups: autologous bone (Group A, n=18), POC-M-click-HA (Group B, n=18), and PLLA-HA (Group C, n=18). All rabbits were fasted for 24 hours, after which time the rabbits were sedated with an injection of 2% sodium pentobarbital (30 mg/kg) and prepared for surgery per standard practice. Rabbits were then placed in a lateral position, and the L4-L5 transverse processes were exposed and removed through anterolateral surgical intervention to reveal the L4/L5 discs. Anterolateral surgical intervention was used in lieu of a conventional surgical method in order to improve access to the target disc and fixation of the vertebral bodies with the steel plate being used. The L4/L5 discs were then resected. After addressing minor bleeding from the dissected ends, the autologous bone, POC-M-click-HA matchstick scaffolds, or PLLA-HA scaffolds were filled into the bone growth site at the L4/L5 discs, and the L4 and L5 vertebrae were fixed into position with screws and connected with a steel plate. The wounds were sututred after being washed and cleaned with a gelatin sponge. All rabbits were dosed with 50,000 U/kg of penicillin intramuscularly for 3 consecutive days to prevent infection. The rabbits were allowed access to food 24 hours after surgery.

### General Observations

At 4, 8, and 12 weeks post-operation, the position of the tissue construct and spinal fusion rate for every specimen was evaluated by three advanced radiologists in a double-blind test adapted from the Suk's system, whereby radiographic and stretching palpitation tests were performed. “Solid union” was defined as an obvious intertebral bone bridge formed when the invertebral range of motion (ROM) on flexion-extension radiographs was <4°. “Probable union” was confirmed when a subtle invertebral bone bridge was formed, but with <4° invertebral ROM on flexion-extension radiographs. “Fusion nonunion” was defined as exhibiting little or no bone formation between vertebra and spine, and a ROM beyond 4° on flexion-extension radiographs. At each point in time, the number of specimens meeting the standards of solid and probable unions in the three groups was recorded, and the fusion rate for each group at a certain time was calculated by equation (2):

$$Fusion rate ( \% ) = \frac{{N}_{t} - {N}_{non}}{{N}_{t}} \times 100 , \qquad ( 2 )$$

wherein Nₜ is the total number of specimens tested and Nₙₒₙ is the number of nonuunion specimens. For each sample, the fusion rate values obtained by the three radiologists were averaged.

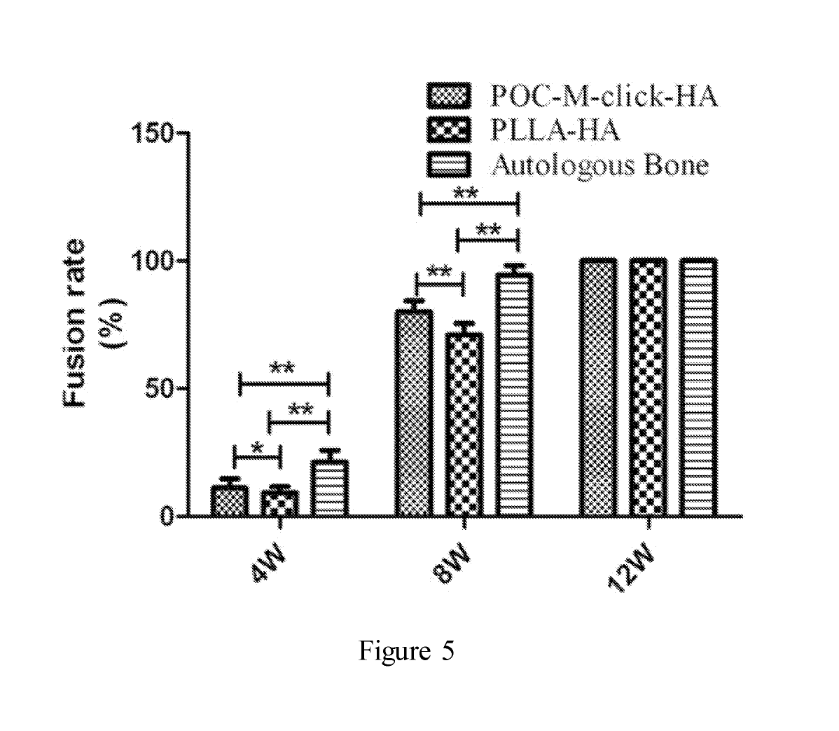

The fusion rates observed in each group according to imaging evaluations are provided in FIG. 5. No migration or breakage of the implant or internal fixation device was observed by X-ray imaging 4 weeks after surgery. No obvious new bone formation was observed, but the implants remained in the disc space at this point in time. The degree of fusion of the autologous bone group (Group A) was 21.3±3.7%. Also, the fusion rates of groups B and C did not differ significantly (p>0.05, FIG. 5) at the 4 week time point. At 8 weeks after operation, although the fusion rate of the autologous bone group (94.4±3.7%) was higher than those of the other two groups (80±4.5% for POC-M-click-HA group and 71.1±4.4% for PLLA-HA group), the fusion rate of the POC-M-click-HA group was still significantly higher than that of the PLLA-HA group. At 12 weeks after surgery, all specimens in the three groups exhibited an invertebral fusion rate of 100% according to palpation testing and micro-CT evaluation. The results of manual palpation examination further confirmed the micro-CT imaging results.

### Biomechanical Testing

The L4-L5 vertebral bodies and intervertebral discs at each time point were removed and stored at −20° C. Before mechanical testing, the specimens were warmed to room temperature and both ends of the specimen were embedded in denture base resin (poly(methyl methacrylate), PMMA) to make sure the plane in which the discs were disposed were vertical relative to the compression direction. The specimens were then fixed on a mechanical tester (Bose Electro Force 3510, USA), and the load was applied at a fixed rate of 0.008 mm/s for compression tests. Values for spinal stiffness and maximum load were recorded.

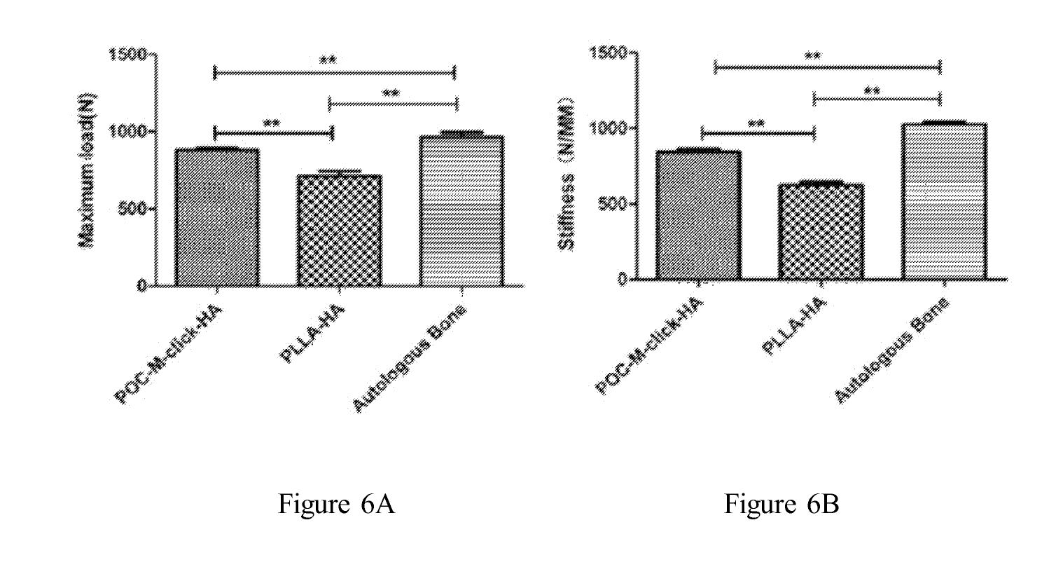

The results for biomechanical testing of the above-identified samples are illustrated in FIGS. 6A and 6B. The mean maximum load of the POC-M-click-HA group (Group B) was 880.0±14.5 N. Although this value is lower than that of the autologous bone group, which demonstrated a mean maximum load of 965.2±38.8 N, it is significantly higher than that of the PLLA group, which demonstrataed a mean maximum load of 712.0±37.5 N (p<0.05). The fused spinal defect treated with POC-M-click-HA scaffolds also possessed much higher stiffness (843.2±22.4 N/mm) than spinal defects treated with PLLA-HA (622.5±28.4 N/mm, p<0.05). The stiffness of the fused spinal defects treated with autologous bone grafts was 1024.3±21.5 N/mm.

### Bone Regeneration and Graft/Scaffold Degradation

Microscopic images of all three groups stained with H&E and Masson's trichrome staining showed that new bone formation was visible at the fusion segment at week 12, indicating that all three approaches induced trabecular bone formation. No significant local inflammation response around and in the implanted materials was found at any of the measured time points. After 4 weeks, the POC-M-click-HA implant was surrounded by more new bone than the PLLA-HA implant, and the POC-M-click-HA material showed partial degradation, leaving behind sporadic cavities. In contrast, less new bone formation surrounding the PLLA-HA implant was observed. Further, very little material degradation of the PLAA-HA implant was observed. After 8 weeks, the POC-M-click-HA composite degraded more, leaving only a small amount of the material inside the new bone. Minimal new bone growth was observed around the PLLA-HA implants at 8 weeks, and material degradation was not readily apparent in the same samples. At week 12, the POC-M-click-HA group demonstrated new bone largely replacing the composite, filling the intervertebral disc space, and connecting the upper and lower vertebral bodies. The implanted PLLA-HA material also showed new bone formation, but with significantly more residual material surrounding the newly formed bone.

Various embodiments of the present invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A method of promoting bone growth comprising:
disposing a scaffold in a bone growth site, the scaffold comprising:
(a) a polymer network formed from the reaction product of (i) citric acid, a citrate, or an ester of citric acid with (ii) a polyol; and
(b) a particulate inorganic material dispersed in the polymer network.

2. The method of claim 1, wherein the particulate inorganic material comprises one or more of hydroxyapatite, tricalcium phosphate, biphasic calcium phosphate, bioglass, ceramic, magnesium powder, magnesium alloy, and decellularized bone tissue particles.

3. (canceled)

4. The method of claim 1, wherein the ester of citric acid comprises triethyl citrate.

5. The method of claim 1, wherein the polyol comprises a C2-C20 α,ω-n-alkane diol or a C2-C20 α,ω-alkene diol.

6. The method of claim 1, wherein the polymer network is formed from the reaction product of (i) citric acid, a citrate, or an ester of citric acid with (ii) a polyol and (iii) an amine, an amide, or an isocyanate.

7. (canceled)

8. (canceled)

9. (canceled)

10. (canceled)

11. (canceled)

12. The method of claim 6, wherein the amine comprises dopamine.

13. The method of claim 1, wherein the polymer network is formed from the reaction product of (i) citric acid, a citrate, or an ester of citric acid with (ii) a polyol and (iii) a polycarboxylic acid or a functional equivalent of a polycarboxylic acid.

14. (canceled)

15. The method of claim 13, wherein the functional equivalent of a polycarboxylic acid comprises a cyclic anhydride or an acid chloride of a polycarboxylic acid.

16. The method of claim 13, wherein the polycarboxylic acid or functional equivalent thereof is ethylenically unsaturated.

17. The method of claim 16, wherein the polycarboxylic acid or functional equivalent thereof comprises maleic acid, maleic anhydride, fumaric acid, or fumaryl chloride.

18. The method of claim 1, wherein the polymer network is formed from the reaction product of (i) citric acid, a citrate, or an ester of citric acid with (ii) a polyol and (iii) an amino acid.

19. (canceled)

20. (canceled)

21. (canceled)

22. (canceled)

23. (canceled)

24. The method of claim 18, wherein the amino acid forms a pendant group of the polymer network.

25. (canceled)

26. The method of claim 1, wherein the polymer network is formed from the reaction product of (i) citric acid, a citrate, or an ester of citric acid with (ii) a polyol and (iii) a catechol-containing species.

27. (canceled)

28. The method of claim 1, wherein the reaction product is a condensation reaction product.

29. The method of claim 1, wherein the polymer network is formed from one or more monomers of Formula (A) and one or more monomers of Formula (B1) or (B2): 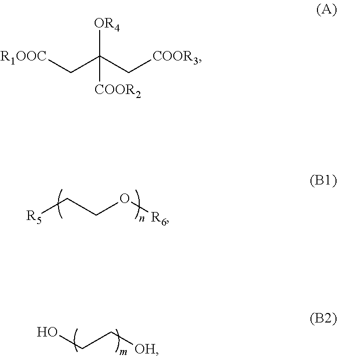
wherein R₁, R₂, and R₃ are independently —H, —CH₃, —CH₂CH₃, or M⁺;
R₄ is —H;
R₅ is —H, —OH, —OCH₃, —OCH₂CH₃, —CH₃, or —CH₂CH₃;
R₆ is —H, —CH₃, or —CH₂CH₃;
M⁺ is a monovalent cation; and
n and m are independently integers ranging from 1 to 20.

30. The method of claim 1, wherein the polymer network is formed from one or more monomers of Formula (A) and one or more monomers of Formula (B4), (B5), or (B6): 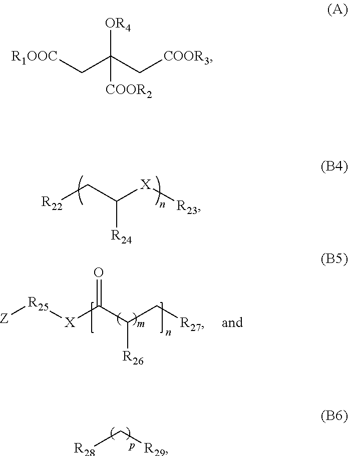
wherein
R₁, R₂, and R₃ are independently —H, —CH₃, —CH₂CH₃, or M⁺;
R₄ is —H;
R₂₂ is —H, —OH, —OCH₃, —OCH₂CH₃, —CH₃, —CH₂CH₃, —NH₂, NHCH₃, —CH₂CH₂NHCH₃, —N(CH₃)₂, or —CH₂CH₂N(CH₂CH₃)₂;
R₂₃ is —H, —CH₃, or —CH₂CH₃, —(CH₃)₂, or —(CH₂CH₃)₂;
R₂₄ is —H or —CH₃;
R₂₅ is —(CH₂)ₐ—, —(CH₂CH₂O)_{b}— or —(CH₂OCH₂)_{b}—;
R₂₆ is —H, —CH₃, or a C2-C20 alkyl;
R₂₇ is —H, —C(O)CH₃, or —C(O)CH₂CH₃;
R₂₈ and R₂₉ are independently —OH or —NH₂;
M⁺ is a monovalent cation;
X and Y are independently —O— or —NH—;
Z is —H, —CH₃, —(CH₃)₂, —(CH₂CH₃)₂, or 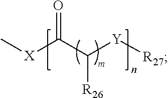
a is an integer from 0 to 20;
b is an integer from 0 to 2000;
n is an integer between 1 and 2000; and
m and p are independently integers ranging from 1 to 20; and
wherein the monomer of Formula (B4) has at least one terminus comprising —OH or —NH₂.

31. The method of claim 30, wherein the polymer network is formed from one or more monomers of Formula (A), one or more monomers of Formula (B4), (B5) or (B6), and one or more monomers comprising one or more alkyne moieties or one or more azide moieties.

32. The method of claim 31, wherein the one or more monomers comprising one or more azide moieties comprises a monomer of Formula (H) or (H′): 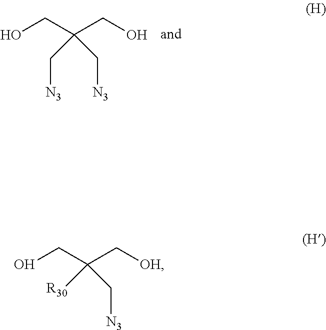
wherein R₃₀ is —CH₃ or —CH₂CH₃.

33. The method of claim 31, wherein the one or more monomers comprising one or more alkyne moieties comprises a monomer of Formula (I1), (I2), (I3), (I4), (I5), or (I6): 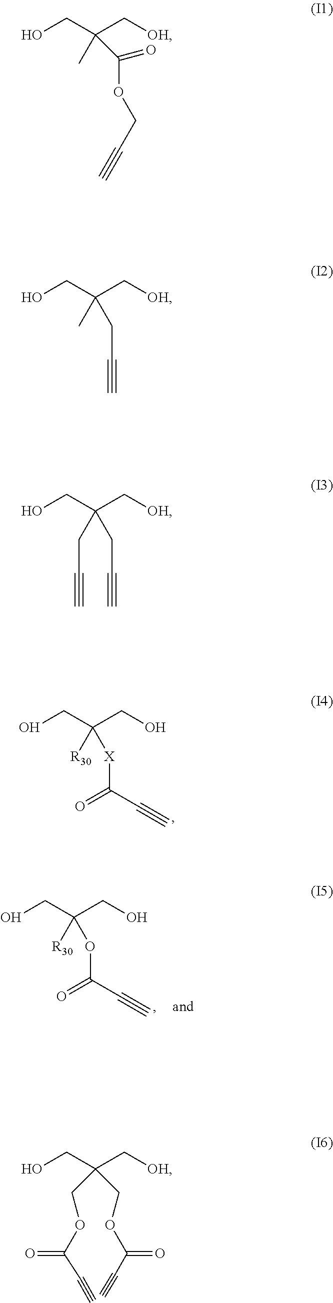
wherein R₃₀ is —CH₃ or —CH₂CH₃; and
X is —NH— or —O—.

34. The method of claim 30, wherein the polymer network is formed from one or more monomers of Formula (A), one or more monomers of Formula (B1) or (B2), one or more monomers comprising one or more alkyne moieties or one or more azide moieties, and one or more monomers comprising a secondary or tertiary amine-containing diol.

35. (canceled)

36. The method of claim 1, wherein the polymer network forms a porous core component.

37. The method of claim 36, wherein the scaffold further comprises a porous shell component surrounding the porous core component.

38. (canceled)

39. The method of claim 36, wherein the polymer network exhibits an average pore size of about 800 nm to about 1000 μm.

40. (canceled)

41. (canceled)

42. (canceled)

43. The method of claim 1, wherein the scaffold is unseeded with a biofactor or cell.

44. (canceled)

45. (canceled)

46. (canceled)
